(19) United States
(12) Patent Application Publication
(10) Pub. No.: US 2007/0098713 A1
(43) Pub. Date: May 3, 2007

(54) Nanoparticle delivery systems and methods of use thereof

(75) Inventors: Gretchen Unger, Chaska, MN (US); Beverly Lundell, Woodbury, MN (US)

Correspondence Address: DARDI & ASSOCIATES, PLLC, 220 S. 6TH ST., SUITE 2000, U.S. BANK PLAZA, MINNEAPOLIS, MN, 55402, US

(73) Assignee: Genesegues, Inc.

(21) Appl. No.: 11/584,044

(22) Filed: Oct. 20, 2006

Related U.S. Application Data
(63) Continuation of application No. 10/410,659, filed on Apr. 8, 2003, now abandoned.
(63) Continuation-in-part of application No. 10/378,044, filed on Feb. 28, 2003.
(60) Provisional application No. 60/394,315, filed on Jul. 8, 2002.
(60) Provisional application No. 60/370,882, filed on Apr. 8, 2002.
(60) Provisional application No. 60/428,296, filed on Nov. 22, 2002.

(51) Int. Cl. A61K 48/00; A61K 39/395; A61K 9/14

(52) U.S. Cl. 424/133.1; 514/44; 424/489; 977/906; 424/423

(57) ABSTRACT

Certain embodiments of the invention relate to the use of small particles in biological systems, including the delivery of biologically active agents. Some embodiments involve using a collection of particles comprising an agent, a surfactant molecule having an HLB value of less than about 6.0 units, and a polymer soluble in aqueous solution, wherein the collection of particles has an average diameter of less than about 200 nanometers, wherein the agent is a protein, carbohydrate, polypeptide, adjuvant, nucleic acid encoding a protein, visualization agent, and/or a marker.

### RELATED APPLICATIONS

This application is a continuation of U.S. patent application Ser. No. 10/410,659, filed Apr. 8, 2003, which claims priority to U.S. Patent Application Ser. Nos. 60/394,315, filed Jul. 8, 2002; 60/370,882 filed Apr. 8, 2002 and 60/428,296, filed Nov. 22, 2002, and which is a continuation-in-part of U.S. patent application Ser. No. 10/378,044, filed Feb. 28, 2003, each of which are hereby incorporated herein by reference.

The Sequence Listing file named “3193.03-US-03.ST25.txt” (32,768 bytes in size) submitted to the U.S. Patent and Trademark Office on Compact Disc-Recordable (CR-R) medium (entitled “031229_{—}1018” and created on Dec. 29, 2003) in compliance with 37 C.F.R. § 1.52(e) is incorporated herein by reference.

### FIELD OF INVENTION

The field of the invention relates to the use of small particles in biological systems, including the delivery of biologically active agents.

### BACKGROUND

Over the past several decades, active and extensive research into the use of small particles in the delivery of therapeutic macromolecules to target cells has generated a number of conventional approaches in the preparation of small particles. Delivery of small particles, however, is complicated by the fact that the body has cells that tend to clear the particles from the body, so that the particles are removed from the body before they reach the target cells that they are intended to affect. Another complicating factor is that conventional particles are often transported into cells into lysosomes, which are vessels in the cells that degrade the particles and their contents so that the efficacy of the therapeutic agents in the particle is reduced.

### SUMMARY OF THE INVENTION

Certain embodiments of the invention relate to the use of small particles in biological systems, including the delivery of biologically active agents. Some embodiments relate to a collection of particles having an agent, a surfactant molecule having an HLB value of less than about 6.0 units, and a polymer, wherein the collection of particles has an average diameter of less than about 100 nanometers as measured by atomic force microscopy of dry particles, wherein the agent is a protein, carbohydrate, polypeptide, adjuvant, nucleic acid encoding a protein, a visualization agent, or a marker.

Some embodiments relate to a method of delivering an agent to an antigen presenting cell by exposing a cell to a collection of particles that have an agent, a surfactant molecule having an HLB value of less than about 6.0 units, a polymer, and a ligand that binds to an antigen presenting cell, wherein the collection of particles has an average diameter of less than about 100 nanometers.

Some embodiments relate to a method of affecting function of a cell, the method comprising exposing the cell to an agent that specifically or preferentially inhibits protein kinase 2 function, e.g., an antisense molecule directed against a CK2 subunit.

Some embodiments relate to a collection of particles having an agent, a surfactant molecule having an HLB value of less than about 6.0 units, and a polymer, with the collection of particles having an average diameter of less than about 50, 100, or 200 nanometers as measured by atomic force microscopy of a plurality of the particles following drying of the particles, wherein the agent is an imaging agent.

Some embodiments relate to a method of delivering an agent to a cell by exposing a cell to a collection of particles that comprises an imaging agent, a surfactant molecule having an HLB value of less than about 6.0 units, and a polymer, wherein the collection of particles has an average diameter of less than about 200, 100, or 50 nanometers as measured by atomic force microscopy of a plurality of the particles following drying of the particles.

Some embodiments relate to a kit having a collection of particles that include an agent, a surfactant molecule having an HLB value of less than about 6.0 units, and a polymer, wherein the collection of particles has an average diameter of less than about 50, 100, or 200 nanometers as measured by atomic force microscopy of a plurality of the particles following drying of the particles, wherein the agent is an imaging agent, with the kit optionally also having instructions for using the collection of particles.

Some embodiments relate to a method of delivering an agent to a cancer cell by exposing a cancer cell to a collection of particles that comprises an agent, a surfactant molecule having an HLB value of less than about 6.0 units, a polymer, an adjuvant, and a ligand that targets to the cancer cell, wherein the collection of particles has an average diameter of less than about 50, 100, or 200 nanometers as measured by atomic force microscopy of a plurality of the particles following drying of the particles.

Some embodiments relate to a collection of coated particles that has particles and a coating, the coating comprising a binder and the particles comprising an agent, a surfactant molecule having an HLB value of less than about 6.0 units, and a polymer, wherein the collection of particles has an average diameter of less than about 50, 100, or 200 nanometers as measured by atomic force microscopy of a plurality of the particles following drying of the particles.

Some embodiments relate to a biocompatible stent associated with or coated with a collection of particles. Some embodiments relate to a method of coating a collection of particles by mixing a binder with a collection of particles, with the particles having an agent, a surfactant molecule having an HLB value of less than about 6.0 units, and a polymer, wherein the collection of particles has an average diameter of less than about 100 nanometers as measured by atomic force microscopy of a plurality of the particles following drying of the particles.

### BRIEF DESCRIPTION OF THE FIGURES

FIG. 1 depicts the relationship of several major immune system cell types to each other;

FIG. 2 is a montage of photomicrographs showing the delivery of nanoparticles to antigen presenting cells, and the resultant uptake and activation of the antigen presenting cells;

FIG. 3 is a montage of photomicrographs showing delivery of nanoparticles to antigen presenting cells, and the resultant uptake and activation of the antigen presenting cells, as evidenced by the migration of the antigen presenting cells;

FIG. 4 is a photomicrograph of nanoparticles visualized using atomic force microscopy;

FIG. 5 is a graph showing tumor regression for cancerous mice treated with nanoparticles targeted to the cancer cells, and used to deliver antigenic proteins to the tumors for activation of the antigen presenting cells and immune system;

FIG. 6 is a montage of photomicrographs of tumor tissue taken from cancer-model mice that spontaneously developed cancer;

FIG. 7 is a graph showing the results of treatments of sensitized mice with nanoparticles;

FIG. 8 depicts the messenger RNA (mRNA) sequence of Protein Kinase CK2 alpha prime (SEQ ID No:1);

FIG. 9 depicts the messenger RNA (mRNA) sequence of Protein Kinase CK2 beta (SEQ ID No:2);

FIG. 10 depicts the messenger RNA (mRNA) sequence of Protein Kinase CK2 alpha (SEQ ID No:3);

FIG. 11 is a montage of photomicrographs showing that tumor-targeted nanoparticles containing 10 nm colloidal gold can be used to enhance target-to-background signal by X-Ray imaging in tumor-bearing tissue;

FIG. 12 is a montage of photomicrographs of tissue sections from the experiment depicted in FIG. 11, and correlates tumor nest location with silver deposits accumulated on gold particles.

FIG. 13 is a montage of photomicrographs showing tenascin nanoparticles containing colloidal gold and PVP nanoparticles containing fluorescent semiconductor quantum dots used in these experiments. The scale is equivalent to 250 nm;

FIG. 14 is a montage of photomicrographs showing uptake, nuclear localization and fluorescence after 5 days in rat neonatal cardiomyocytes of PVP nanoparticles containing green fluorescent semiconductor quantum dots from FIG. 3;

FIG. 15 is a chart describing results of a FACS experiment using rat bone marrow cells and GMCSF nanoparticles containing non-bleaching semiconductor dots; At low dosing, cells capable of potocytosing GMCSF capsules were detectable by FACS; At higher dosing, subsequent proliferation diluted signal;

FIG. 16 is a montage of charts describing cytotoxicity of a nanoparticle containing an anti-proliferative antisense construct at anti-tumor dosing levels; A dose response for paclitaxol cytoxicity against both proliferative and quiescent coronary artery endothelial and smooth muscle cells showed that wound endothelial cells are the most sensitive to paclitaxol.

### DETAILED DESCRIPTION

Embodiments of the invention are described herein that relate to the materials and methods for use of small particles, e.g., as markers for antigen presenting cells (APCs), for drug release from coatings, and as visualization tools. The small particles may be nanoparticles with a diameter of less than about 100 nm, or less than about 50 nm, and may include an agent, a lipophilic surfactant having a hydrophilic-lipophilic balance (HLB) of less than about 6, a polymer, and, optionally, ligands specific for targets on cells or tissues. Nanoparticles may also be made and used in metastable forms, enabling eventual release of nanoparticle contents. The term nanoparticle encompasses nanocapsules, nanospheres, nanotoroids, nanocolloid and various geometries.

The use of nanoparticles is generally advantageous compared to larger sized particles because the nanoparticles enter into the cell via caveolar potocytosis, and thereby avoid the fate of larger particles, which are degraded in lysosomes. Such nanoparticles are further distinguished by their capacity for penetration across tissue boundaries, such as the epidermis and endothelial lumen. Further, the use of cell-specific ligands on the nanoparticles has been shown to result in cell-specific delivery, see commonly owned and assigned U.S. patent application Ser. No. 09/796,575 filed Feb. 28, 2001 and 10/378,044, filed Feb. 28, 2003. Some embodiments of the invention are directed to using nanoparticles for the delivery of agents to dendritic cells. Suitable agents may be used as, e.g., markers, research tools, and antigens for immunostimulation.

Antigenic agents that are delivered to dendritic cells are processed by the cells and presented to other components of the immune system so that the immune system is trained to respond to the antigen. A cellular component that possesses that antigen would then be rejected by the immune system. For example, if an antigen from a polio virus vaccine is delivered to a dendritic cell, then the immune system may become trained to recognize that antigen. As a result, if polio virus is subsequently introduced to the body, the immune system will attack both the free virus and infected cells that now express viral markers on their surface because the virus is inside. Many vaccines work on this principle of immune system stimulation. Embodiments are disclosed herein that include, for example, delivering antigens to antigen presenting cells (APCS), including, for example, dendritic cells.

Another way to train the immune system is to introduce potentially antigenic materials into the body along with another material that activates the immune system, usually by triggering an inflammatory response. Adjuvants work in this manner. For example, a material that is introduced into a body with an adjuvant is recognized as being antigenic because, in part, the adjuvant has activated portions of the immune system. Some vaccines use adjuvants to enhance their effectiveness. Embodiments are disclosed herein that include using adjuvants in nanoparticles to trigger immune responses.

Another aspect of the immune system is its role in cancer. Cancer cells that grow into tumors are typically able to evade the immune system. If the immune system can be trained to recognize the cells better, however, then the immune system can attack the cancer. One approach for training the immune system is to deliver antigenic materials to the cancer cells so that the cells become recognizable to the immune system. For example, nanoparticles may be loaded with plasmids that encode bacterial proteins and delivered to cancer cells. The cancer cells express the bacterial protein and are then recognizable by the immune system. Another approach for training the immune system is to introduce factors that trigger the immune system, e.g., adjuvants, into the region of cancerous cells so that the immune system recognizes the cells. Embodiments are set forth herein that include nanoparticles having such factors, and methods of introducing them into, near, or in the region of cancer cells.

Nanoparticles and Methods of Making

The manufacture and process chemistry of nanoparticles is described in detail in U.S. patent Ser. No. 09/796,575 filed Feb. 28, 2001, and Ser. No. 10/378,044 Feb. 28, 2003. In brief, a suitable method of making a nanoparticle is to form a dispersion of micelles by forming a plurality of surfactant micelles, wherein the plurality of surfactant micelles comprises a surfactant interfacing with a bioactive component, wherein the surfactant can have a hydrophile-lipophile-balance (HLB) value of less than about 6.0 units. Then the surfactant micelles are dispersed into an aqueous composition, wherein the aqueous composition comprises a hydrophilic polymer so that the hydrophilic polymer associates with the surfactant micelles to form stabilized surfactant micelles. The stabilized micelles may have an average diameter of less than about 200 or 100 or 50 nanometers. Non-ionic surfactants may alternatively be used. The stabilized surfactant micelles may be precipitated, e.g. using a cation, to form nanoparticles having an average diameter of less than about 200 or 100 or 50 nanometers, as measured by atomic force microscopy of the particles following drying of the particles. Moreover, in some embodiments, the particles may be incubated in the presence of at least one cation. Embodiments wherein nanoparticles have a diameter of less than 200 or 100 or 50 nm, including all values within the range of 5-200 nm, are contemplated. Following incubation, particles are collected by centrifugation for final processing. Particles show excellent freeze-thaw stability, stability at −4° C., mechanical stability and tolerate speed-vacuum lyophilization. Stability is measured by retention of particle size distribution and biological activity. Drug stocks of 4 mg/ml are routinely produced with 70-100% yields.

The term precipitate refers to a solidifying or a hardening of the biocompatible polymer component that surrounds the stabilized surfactant micelles. Precipitation also encompasses crystallization of the biocompatible polymer that may occur when the biocompatible polymer component is exposed to the solute. Examples of cations for precipitation include, for example, Mn2+, Mg2+, Ca2+, Al3+, Be2+, Li+, Ba2+, Gd3+.

The amount of the surfactant composition in some embodiments may range up to about 10.0 weight percent, based upon the weight of a total volume of the stabilized surfactant micelles. Typically however, the amount of the surfactant composition is less than about 0.5 weight percent, and may be present at an amount of less than about 0.05 weight percent, based upon the total weight of the total volume of the stabilized surfactant micelles. A person of ordinary skill in the art will recognize that all possible ranges within the explicit ranges are also contemplated.

A nanoparticle may be a physical structure such as a particle, nanocapsule, nanocore, or nanosphere. A nanosphere is a particle having a solid spherical-type structure with a size of less than about 1,000 nanometers. A nanocore refers to a particle having a solid core with a size of less than about 1,000 nanometers. A nanocapsule refers to a particle having a hollow core that is surrounded by a shell, such that the particle has a size of less than about 1,000 nanometers. When a nanocapsule includes a therapeutic macromolecule, the therapeutic macromolecule is located in the core that is surrounded by the shell of the nanocapsule.

Embodiments herein are described in terms of nanoparticles but are also contemplated as being performed using nanocapsules, the making and use of which are also taught in commonly assigned copending application Ser. No. 09/796,575, filed Feb. 28, 2001, which teaches methods for making particles having various sizes, including less than about 200 nm, from about 5-200 nm, and all ranges in the bounds of about 5 and about 200 nm. The same application teaches how to make s50 nanoparticles. An s50 nanoparticle is a nanoparticle that has an approximate diameter of less than about 50 nm.

The bioactive component, in some embodiments, may be partitioned from the hydrophilic polymer in the nanoparticles, and may be, for example, hydrophobic or hydrophilic. Bioactive components may include proteins, peptides, polysaccharides, and small molecules, e.g., small molecule drugs. Nucleic acids are also suitable bioactive components for use in nanoparticles, including DNA, RNA, mRNA, and including antisense RNA or DNA. When nucleic acids are the bioactive component, it is usually desirable to include a step of condensing the nucleic acids with a condensation agent prior to coating or complexing the bioactive component with the surfactant, as previously set forth in U.S. patent application Ser. No. 09/796,575, filed Feb. 28, 2001.

A wide variety of polymers may be used as the biocompatible polymer, including many biologically compatible, water-soluble and water dispersible, cationic or anionic polymers. Due to an absence of water diffusion barriers, favorable initial biodistribution and multivalent site-binding properties, hydrophilic polymer components are typically useful for enhancing nanoparticle distribution in tissues. However, it will be apparent to those skilled in the art that amphoteric and hydrophobic polymer components may also be used as needed. The biocompatible polymer component may be supplied as individual biocompatible polymers or supplied in various prepared mixtures of two or more biocompatible polymers that are subsequently combined to form the biocompatible polymer component. Though descriptions of the present invention are primarily made in terms of a hydrophilic biocompatible polymer component, it is to be understood that any other biocompatible polymer, such as hydrophobic biocompatible polymers may be substituted in place of the hydrophilic biocompatible polymer, in accordance with the present invention, while still realizing benefits of the present invention. Likewise, it is to be understood that any combination of any biocompatible polymer may be included in accordance with the present invention, while still realizing benefits of the present invention.

The Immune System

The immune system provides a defense against infectious agents. Infectious agents include four major categories: bacteria, fungi, parasites, and viruses. Viruses have a core of RNA or DNA that is sometimes surrounded by a protein coat referred to as a capsid. Some viruses have RNA or DNA surrounded by an envelope of lipids, proteins, and/or glycoproteins. Examples of viruses include acquired immune deficiency syndrome (AIDS), poliomyelitis, chickenpox, smallpox, measles, hepatitis, and herpes. A parasite is an animal or plant that lives on or in another animal or plant of a different type and feeds from it. Parasites include, for example, Leishmania, Acanthamoeba, Amoebae, flagellates, Giardia, Entamoeba, Cryptosporidium, Isospora, Balantidium, Trichomonas, Plasmodium, Trypanosoma, Naegleria, and Toxoplasma. Plasmodium Fungi include the two broad groups of fungi: yeasts and moulds. The nuclei of all fungi have a nucleolus and chromosomes. As in other eukaryotic organisms, fungi have mitochondria, ribosomes, and centrioles. The cell walls of fungi typically consist of chitin, chitosan, glucan, mannan, and other components. Bacteria are microorganisms that do not have internal cell membranes. Bacteria include Salmonella, Vibrio cholerae, Clostridium perfringens, Shigella, enterics, Streptococcus, Clostridium botulinum, Staphylococcus aureus, and the enterovirulent escherichia coli group.

The immune system can identify and attack foreign substances, which are substances that are not native to the body. The lack of immune system reactivity against the body is called self-tolerance. The immune system's reaction against a foreign substance is called an immune response. A substance that induces an immune response is called an antigen. The specific portion of the antigen that induces the immune response is the epitope.

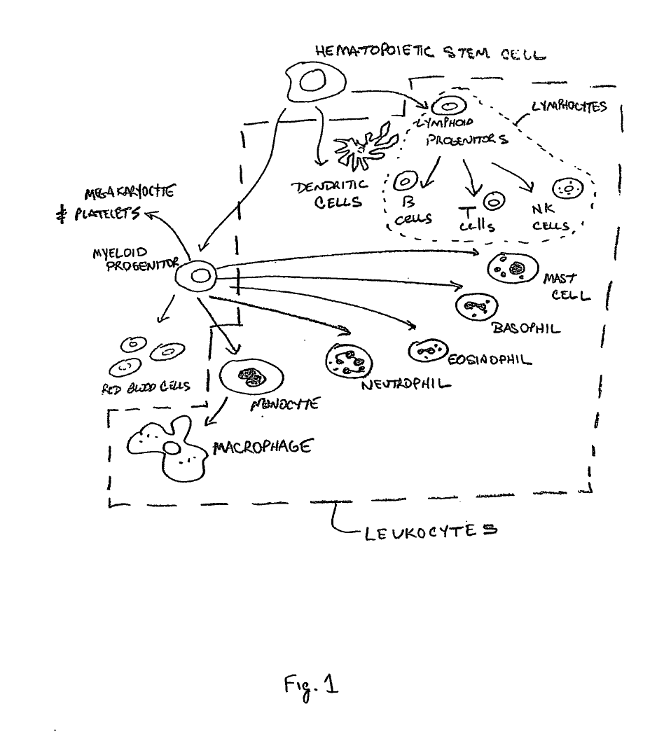

An immunogenic response can be mounted by the innate immune system or the humoral immunity system (the humoral system involves the generation of antibodies). FIG. 1 shows the lineage and types of many of the immune system's cells, including the innate and humoral immune systems. These cell types are recognizable by using markers and morphological information. The lymphoid progenitor cell differentiates into T cells, B cells, and natural killer cells. Dendritic cells are typically characterized by many long membrane extensions. They are found in both lymphoid and nonlymphoid tissues, as well as in the blood and lymph. Dendritic cells share most features in common, but exhibit some variation according to their location: thymus, lymphoid tissue, dermis (dermal dendritic cells, DDC), skin (termed Langerhans cells), veiled cells in the lymph, and blood (blood dendritic cells).

An aspect of the humoral immunity system function involves the processing of antigens so that they become complexed with major histocompatibility complex (MHC) molecules. T-cells have receptors that can subsequently bind the complexed antigens. The cells that display antigen-MHC complexes for recognition by T cells are called antigen presenting cells (APCs). A limited number of cell types can serve as APCs. These are generally recognized as: B lymphocytes, macrophages and monocytes, dendritic cells (including Langerhans), and some epithelial and skin-derived endothelial cells. Antigens, e.g., proteins, RNA, or DNA, introduced into APCs will typically be processed so that the APCs will present the antigens with the MHC complex. As a result, the immune system becomes trained to respond to that antigen and to destroy cells that present it. Antigen presentation by dendritic cells is reviewed in Guermonprez et. al (2002), Ann. Rev. Immunol. 20:621-27.

The ability of an agent to induce an immune response is sometimes enhanced by use of an adjuvant. Adjuvants are agents that augment, or modulate the immune response at either the cellular (innate immune) or humoral level. The classical agents (Freund's adjuvant, BCG, Corynebacterium parvum, and the like) contain bacterial antigens. Some are endogenous (e.g., histamine, interferon, transfer factor, tuftsin, interleukin-1). Their mode of action is either non-specific, resulting in increased immune responsiveness to a wide variety of antigens, or antigen-specific, i.e., affecting a restricted type of immune response to a narrow group of antigens. Other adjuvants include nickel, Montanide ISA, Ribi Adjuvant System, Syntex Adjuvant Formulation, aluminum salt adjuvants, nitrocellulose-adsorbed antigen (slow-release), Immune-stimulating complexes, e.g., antigen modified saponin/cholesterol micelles, and GerbuR adjuvant.

A vaccine is a substance that causes a body to produce antibodies as a step towards developing immunity. Thus, for example, a polio vaccine causes the body to develop immunity to the polio virus. Vaccines can involve introducing antigenic materials into a body so that they are taken up by APCs and processed into MHC complexes. Administration of antigenic compounds, particularly genetic constructs, for the purpose of therapeutic immune stimulation against an invading or infectious pathogen or a tumor is a desirable goal because they are often easier to manufacture and transport, and are more stable than conventional vaccines based on traditional protein antigens. The genetic construct, e.g., a plasmid, can have nucleic acids that encode a sequence for an antigenic protein. The plasmid is transcribes RNA, which is translated into a protein, which has antigens. Conceptually, the target organism's immune system recognizes an antigen, and generates humoral (antibody)- and/or cell-mediated immune responses. Further, genetic constructs overcome the need to cultivate dangerous infectious agents and provide a possibility to vaccinate against multiple pathogens in a single administration, or dose.

However, these promising strategies have been limited, in the past, by inadequate delivery of antigenic gene constructs to the APCs, despite much effort at optimization of vector, dosing schedule and adjuvants (reviewed in Vol. 43 (2000) Advanced Drug Deliv. Reviews, Schultz et. al and Weeratna et al. (2000), Intervirology 43: 197-226). There is also a need for improved delivery of current conventional vaccines, in an effort to limit inappropriate activation of the target organism's immune system against antigen or adjuvant and immune complexes inadvertently deposited in an inappropriate site (Singh et. al (2002), Pharm Res. 19(6):715-28). Finally, there is a need to therapeutically deliver genetically constructed material that is inherently inflammatory in a way that can be regulated for the benefit of the application and is not toxic (Krieg et. al (2002), Ann Rev. Immunol. 20:700-60).

Materials and methods for uses of nanoparticles as set forth herein address these needs, and describe specific delivery of antigenic molecules, or nucleic acid sequences that encode antigenic molecules, to cell and tissue-specific targets, including APCs, using nanoparticles. In another embodiment, particles are manufactured to be larger than 50 nm, enabling a period of extracellular dissolution and release of the particle cargo for generalized immunostimulation. This combined approach of using readily-assembled particles with ligand-based targeting enables a method of rational design for drug delivery based on cell biology and regional administration.

The immune system is modeled in a variety of ways, so that research tools, markers, and vaccine may be evaluated. Many aspects of immune system function may be modeled using cultured cells. Cell culture is useful for, e.g., the study of antigen processing, immune cell function, APC maturation states, and markers. The complex interrelation of cells, however, is difficult to model using isolated cells that have been mixed in a petri dish. Such mixing disrupts the cellular interrelationships so that some aspects of cellular function are not well modeled. Thus an organ culture is used for some experiments herein. A preferred system is the punch biopsy system, wherein sections of epidermis and dermis are taken from an animal using a biopsy tool using aseptic technique. These portions of living tissue may be stored for later use by freezing in media containing 20% fetal calf serum and 10% DMSO. At the desired time of the assay, the tissue may be thawed and cultured at an air-water interface to simulate the situation where epidermis is ‘dry’ and dermis is ‘wet’ by culturing biopsies on a platform of 80 mesh stainless steel screen in contact with media containing 20% fetal calf serum in standard, sterile organ culture dishes (Fisher). Media is changed in these dishes every other day and viable tissue can be maintained in this way for at least 7 days (Unger et. al, (2003).

Punch biopsies from skin contain both dermal dendritic cells and epidermal Langerhans cells, as does intact skin on a living animal; both cell types are dendritic cells. Langerhans cells are present in the tissue of mammals, including humans. Agents applied to the skin of animals that have a hydrophobic nature and are generally under a molecular weight of about 500, penetrate the epidermis and encounter the Langerhans cells (Transdermal and topical delivery systems, Ghosh, T. ed., (1997); Interpharm, Inglewood, Colo.). Thus the delivery of antigens to the immune system is greatly simplified by ready access to these cells. Nanoparticles described herein readily penetrate the skin and may be taken up and processed by the immune system. It is believed that the small size of the nanoparticles allows them to penetrate deep into the epidermis, where they are available to interact with the Langerhans cells.

Targeting of APCs with Nanoparticles

APCs, including leukocytes, may be targeted by making nanoparticles having ligands that recognize targets on the APCs. Observations suggest that targeting the APCs with nanoparticles stimulate the dendritic cells and effectively activate the immune system. The nanoparticles bind the targets, are internalized by the cells, and release the nanoparticle's contents into the cell. Targets are preferably receptors that are internalized into the cells by a caveolar pathway. Many suitable targets, including receptors, are known to exist on APCs. Examples of such receptors include, for example, the following receptors, or receptors for: E-selectin, CD3, CD 4, CD8, CD11, CD 14, CD 34, CD 123, CD 45Ra, CD64, E-cadherin, ICAM-1, interleukins, interferons, tumor necrosis factors, E-cadherin, Fc, MCH, CD 36 and other integrins, chemokines, Macrophage Mannose receptor and other lectin receptors, B7, CD's 40, 50, 80, 86 and other costimulatory molecules, Dec-205, scavenger receptors and toll receptors, see also Guermonprez et al. (Annu. Rev. Immunol., 2002). Dendritic cells are considered to be highly effective APCs for initiating MHC-restricted and innate immune responses. Their biology and role in many health and disease states is reviewed in Lipscomb et. al (2001), Physiol. Rev. 82:97-130. Alternatively, particles, e.g., nanoparticles, may be taken up by phagocytosis of macrophages, where antigenic contents of the nanoparticles will be processed and presented as antigens.

An example of a ligand for targeting an APC or a leukocyte is E-selectin. E-selectin plays an active role in inflammatory activation where activated vascular endothelial cells upregulate E-selectin as a receptor for leukocytes (reviewed in U.S. Pat. No. 5,962,424 and references incorporated therein, such references being hereby incorporated by reference). Example 1 demonstrates materials and methods for using E-selectin as a ligand for targeting APCs or leukocytes.

Dendritic cells, and certain other APCs, participate in the innate immune system, which is deployed throughout the body. The innate immune system sometimes initiates and mounts an inflammatory response to an infectious or antigenic agent. APCs participate in such responses and present antigens to T cells for further processing and immunity development. Some embodiments include making nanoparticles having ligands that specifically or preferentially target APCs, including leukocytes and dendritic cells.

Nanoparticles for Delivery of Agents

Nanoparticles may be used to deliver a wide variety of agents, including bioactive, diagnostic, and visualization agents, see commonly owned and assigned U.S. patent application Ser. No. 09/796,575 filed Feb. 28, 2001 and Ser. No. 10/378,044, filed Feb. 28, 2003. Agents include markers, visualization agents, fluorescent particles, adjuvants, dendritic cell maturation factors, and antigens, e.g. proteins foreign to the immune system receiving the proteins.

A suitable use of small particles is to associate a bioactive agent with the particle, e.g., by association bonds (e.g., Au:S), by chelation, or by adsorption. The particle can then be administered to the patient. Such approaches can be plagued by premature release of the bioactive agent. For example, the agent can decomplex from the particle and be released before the particle reaches a suitable target cell. The particles interact with many proteins, e.g., albumin in blood, and thus the bioactive agents are given many opportunities to exchange the small particle for a cell or protein. The dissociation constant is a measure of the propensity of an agent for exchange: a small dissociation constant indicates a low propensity, and a large constant indicates a ready propensity for exchange.

Some approaches have addressed the issue of premature dissociation by covalently attaching the bioactive agent to a particle. But covalent attachment can create heterogeneous structures difficult to characterize and purify and more importantly, can destroy or reduce the bioactivity of the agent. Further, the kinetics of a solid-solution phase interaction for an agent bonded to a solid particle are much less favorable than the kinetics for a solution-solution reaction for an agent not covalently bonded. For example, the covalent attachment of bioactive molecules to a linker that has a molecule for forming an attachment bond to a surface is described in PCT application WO 01/91808, filed May 31, 2001 by Kotov. Kotov includes descriptions of linkers that have thiol groups that complex to particles with gold surfaces.

In contrast to other approaches, the use of nanoparticles as described herein allows for agents to be released after being taken up by cells. The nanoparticles may be made to be taken up with a high affinity and rate so that associated biological agents are delivered to cells, and not released too soon. The agents are typically tightly bound in nanoparticles until they are released by intracellular processes. The agents are not covalently bound so that they do not thereby suffer a loss of activity.

In some embodiments, nanoparticles may be used to deliver antigenic proteins, e.g., non-native proteins, proteoglycans, polysaccharides, or nucleic acids. Antigenic proteins are proteins that evoke a humoral immune response from an immune system to which they are delivered. For example, a bacterial protein is not antigenic in a bacteria that is in a petri dish, but it is antigenic when it is delivered into a mammal. For some applications, the nanoparticles deliver the biological agent to APCs. The antigenic function of the protein may be used in the context of vaccines and immune system research tools. APCs, e.g., dendritic cells, that receive nanoparticles process the agents in the nanoparticles and present antigenic portions to other immune system cells, so that the immune system in a body generates antibodies against the antigen, and to other agents that also express the antigen. Alternatively, nucleic acids that encode antigenic agents may be delivered to in APCs, where the antigens are expressed and then processed.

Certain embodiments are nanoparticles that contain antigens, or nucleic acids that encode antigens, that are characteristic of an infectious agent, e.g. a virus. After an APC has taken up the antigen and generated and immune response against antigen, the host body will be resistant to infection by the infectious agent. Many antigenic portions of infectious agents are known, and any such portions may be used if they generate sufficient antibody titers. In general, proteins that are foreign to a body are antigenic. For example, bacterially derived proteins are highly antigenic. Thus some embodiments of the invention include identifying an infectious agent and delivering a proteinacious and/or antigenic portion of the infectious agent to a cell using a nanoparticle.

Nucleic acids can be incorporated into vectors, and the vectors may be associated with nanoparticles. As used herein, a vector is a replicon, such as a plasmid, phage, or cosmid, into which another nucleic acid segment may be inserted so as to bring about replication of the inserted segment. Vectors of the invention typically are expression vectors containing an inserted nucleic acid segment that is operably linked to expression control sequences. An expression vector is a vector that includes one or more expression control sequences, and an expression control sequence is a DNA sequence that controls and regulates the transcription and/or translation of another DNA sequence. Expression control sequences include, for example, promoter sequences, transcriptional enhancer elements, and any other nucleic acid elements required for RNA polymerase binding, initiation, or termination of transcription. With respect to expression control sequences, “operably linked” means that the expression control sequence and the inserted nucleic acid sequence of interest are positioned such that the inserted sequence is transcribed (e.g., when the vector is introduced into a host cell). For example, a DNA sequence is operably linked to an expression-control sequence, such as a promoter when the expression control sequence controls and regulates the transcription and translation of that DNA sequence. Suitable expression control sequences for humans and other mammals are known in the art. For example, U.S. Pat. Nos. 4,273,875, 4,304,863, 4,349,629, 4,403,036, and 4,419,450 disclose various aspects of plasmids. U.S. Pat. Nos. 4,332,901, 4,356,270 and 4,362,867 disclose various recombinant cDNA construction methods and U.S. Pat. No. 4,363,877 discloses recombinant DNA transfer vectors. U.S. Pat. No. 4,336,336 discloses a fused gene and a method of making the same. The term “operably linked” includes having an appropriate start signal (e.g., ATG) in front of the DNA sequence to be expressed and maintaining the correct reading frame to permit expression of the DNA sequence under the control of the expression control sequence to yield production of the desired protein product. Examples of vectors include: plasmids, adenovirus, Adeno-Associated Virus (AAV), Lentivirus (FIV), Retrovirus (MoMLV), and transposons.

Diagnostics, Imaging, and Visualization Agents

Nanoparticles may also incorporate visualization agents. Visualization agents are materials that allow the nanoparticles to be visualized after exposure to a cell or tissue. Visualization includes imaging for the naked eye, as well as imaging that requires detecting with instruments or detecting information not normally visible to the eye, and includes imaging that requires detecting of photons, sound or other energy quanta. Examples include stains, vital dyes, fluorescent markers, radioactive markers, enzymes or plasmid constructs encoding markers or enzymes. Many materials and methods for imaging and targeting that may be used in nanoparticles are provided in the Handbook of Targeted delivery of Imaging Agents, Torchilin, ed. (1995) CRC Press, Boca Raton, Fla.

Visualization based on molecular imaging typically involves detecting biological processes or biological molecules at a tissue, cell, or molecular level. Molecular imaging can be used to assess specific targets for gene therapies, cell-based therapies, and to visualize pathological conditions as a diagnostic or research tool. Imaging agents that are able to be delivered intracellularly are particularly useful because such agents can be used to assess intracellular activities or conditions. Imaging agents must reach their targets to be effective; thus, in some embodiments, an efficient uptake by cells is desirable. A rapid uptake may also be desirable to avoid the RES, see review in Allport and Weissleder, Experimental Hematology 1237-1246 (2001).

Further, imaging agents preferably should provide high signal to noise ratios so that they may be detected in small quantities, whether directly, or by effective amplification techniques that increase the signal associated with a particular target. Amplification strategies are reviewed in Allport and Weissleder, Experimental Hematology 1237-1246 (2001), and include, for example, avidin-biotin binding systems, trapping of converted ligands, probes that change physical behavior after being bound by a target, and taking advantage of relaxation rates. Examples of imaging technologies include magnetic resonance imaging, radionuclide imaging, computed tomography, ultrasound, and optical imaging.

Approaches to the targeting of imaging agents involve the use of various conjugation strategies. Such strategies are described, for example, in Nie and Emory (1997); Bruchez et al. (1998); Schreder et al. (2000); Micic et al. (1997); Heath, J (1998); Frechet, J (1994); Grayson and Frechet (2001); Lewin et al. (2000), and Bulte et al. (2001). These strategies may be adapted as appropriate for use with nanoparticles.

Nanoparticles as set forth herein may advantageously be used in various imaging technologies or strategies, for example by incorporating imaging agents into nanoparticles. Many imaging techniques and strategies are known, e.g., see review in Allport and Weissleder, Experimental Hematology 1237-1246 (2001); such strategies may be adapted to use with nanoparticles. Suitable imaging agents include, for example, fluorescent molecules, labeled antibodies, labeled avidin:biotin binding agents, colloidal metals (e.g., gold, silver), reporter enzymes (e.g., horseradish peroxidase), superparamagnetic transferrin, second reporter systems (e.g., tyrosinase), and paramagnetic chelates. Advantages of nanoparticles less than about 100 nm or 50 nm in diameter include for example, the ability of the nanoparticles to be readily delivered and taken up by cells.

Compared to imaging agents that are merely conjugated to a targeting molecule, nanoparticles can increase signal-to-noise ratio by delivering larger imaging agent loads per uptake event resulting in higher amplification. Many imaging agents may be loaded into a particle having a targeting molecule (e.g., tenascin), which passes into a cell via a single uptake event (i.e., caveolar uptake in the case of nanoparticles of less than about 100 nm or 50 nm). In contrast, only a single imaging agent linked to a targeting molecule would be taken up by the same event. Since the internalization, intracellular transport, and recycling of cell surface receptors often requires significant turnaround time, the resultant direct uptake of signal molecules by a cell is slower than the uptake of signal molecules with a nanoparticle.

Magnetic resonance imaging contrast agents may also be used in nanoparticles. Examples of Magnetic resonance imaging contrast agents include, but are not limited to, 1,4,7,10-tetraazacyclododecane-N,N′,N″N′″-tetracetic acid (DOTA), diethylenetriaminepentaacetic (DTPA), 1,4,7,10-tetraazacyclododecane-N,N′,N″,N′″-tetraethylphosphorus (DOTEP), 1,4,7,10-tetraazacyclododecane-N,N′,N″-triacetic acid (Do3A) and derivatives thereof (see U.S. Pat. Nos. 5,188,816, 5,219,553, and 5,358,704). X-Ray contrast agents may also be incorporated into nanoparticles, which may be delivered to a patient, tissue, or cell. X-ray contrast agents already known in the art include a number of halogenated derivatives, especially iodinated derivatives, of 5-amino-isophthalic acid.

Clinical imaging is of increasing helpfulness in clinical and research settings, e.g., as reviewed by Acharya et al., Computerized Medical Imaging and Graphics, 19(1): 3-25 (1995). Current uses include laboratory medicine, surgery, radiation therapy, nuclear medicine, and diagnostic radiology. Nanoparticles may be loaded with agents that enhance these processes, for example by enhancing contrast, or delivering agents to cells that allow for visualization with such techniques.

Diagnostic kits may also be prepared that comprise nanoparticles and suitable imaging agents, as well as, optionally, diagnostic tools for deliveries of the particles and agents, and instructions for a diagnostic use. Such nanoparticles may be less than about 500, 300, 100, or 50 nm, and may be made with surfactants having an HLB value of less than about 6. For example, a kit may comprise nanoparticles that comprise imaging agents and ligands that are targeted to bind to target molecules. The nanoparticles can be administered to, for example, a patient, a body portion, a sample, a specimen, or a test apparatus. The targeting molecule, e.g., an antibody or ligand, becomes associated with a target molecule, e.g., to indicate its presence. The imaging agent is associated with the nanoparticle and may be visualized to detect the nanoparticle and, by implication, the target molecule. Such kits are useful for clinical, medical, and research uses. For example, a kit may be made to image molecules present on a histology slide, or a thin section. The kit may further include instructions for use. Instructions may be, for example, an insert, a label, on packaging, a brochure, a handout, a pamphlet, a web page, or in written or electronic form, including posting on internet sites or intranet locations. Instructions may provide general information for use of the kit, and also provide, for example, information on targets, disease states, and/or targeting molecules. Target molecules may be used, for example, that indicate a disease state, a pathology, or a test result, and may provide qualitative or quantitative indicia.

For example, many peptides have been developed that are specific to certain molecules cell types, and/or tissue types; such peptides may be used a targeting molecules on nanoparticles to target the nanoparticles and their contents, see e.g., U.S. Pat. Nos. 6,528,481, 6,353,090, 6,303,573, and 6,232,287. A peptide is used broadly to mean a linear, cyclic or branched peptide, peptoid, peptidomimetic, or the like. Methods for identifying peptides suitable for targeting are also known, see e.g., U.S. Pat. Nos. 6,306,365, 6,296,832 and 6,232,287.

Also, nanoparticles may be made with antibodies that recognize antigens, including antigens that are diagnostic of a condition in a patient, body, sample, specimen, or test. Or ligands that bind to the antigens may be used. Antigens may thus be detected to determine if a particular molecule is present.

Danger Signals and Adjuvants and Maturation Factors

The types of antigens or infectious agents that stimulate a dendritic cell response are sometimes referred to as danger signals. Danger signals have a role in initiating dendritic cell maturation and subsequent migration to regional lymph nodes. The presentation of danger signals to dendritic cells at about the same time as the presentation of a potentially antigenic agent increases the likelihood that the agent will elicit an antigenic response and is normally expected to increase the strength of that response.

A variety of strategies for presenting danger signals concomitant with nanoparticle presentation to the cell may be used. These include incorporating the danger signals into a nanoparticle and exposing the nanoparticle to dendritic cells. The nanoparticle may also contain various agents, e.g., antigenic materials, or imaging materials. For example, a nanocapsule may be made that has an antigenic agent in its interior and a ligand and danger signal on its exterior. Alternatively, nanoparticles having antigenic agents may be mixed with nanoparticles having danger signals, and the mixture administered to a cell, body, or culture. Alternatively, nanoparticles having an antigenic agent may be administered before or after the administration of a danger signal. Adjuvants possibly serve as danger signals in some circumstances so that adjuvants may be substituted for danger signals. Alternatively, danger signals may be incorporated into nanoparticles; the nanoparticles may be made and administered so as to be taken up by cells and internalized, and to degrade in the extracellular space.

Further, factors that cause the maturation of APCs, e.g., dendritic cells, may also be used in conjunction with nanoparticles, e.g., by incorporation therein. Maturation factors include the chemokines that APCs, e.g., dendritic ells, use as cues for migration. Many such factors are known, e.g., as in Lipscomb and Masten, Physiol. Rev. 2002, Janeway and Medshitov, Ann. Rev. Immunology 2002 and Guermonprez et al., Ann. Rev. in Immunology 2002.

Danger signal receptors and maturation receptors are all potential targets for nanoparticles, which may be made with ligands that bind the receptors. Further, agents that activate the receptors may be used to enhance dendritic cell responses to nanoparticles, e.g., by making nanoparticles with receptor-activating agents. Alternatively, agents that activate such receptors may be administered before, after, or simultaneously with nanoparticles having antigenic materials.

At least five types of surface receptors on dendritic cells have been reported to trigger dendritic cell maturation: i) Toll-like receptors (TLR), ii) cytokine receptors, iii) TNF-receptor family members, iv) FcR, and v) sensors for cell death, see Guermonprez et al., (Annu. Rev. Immunol. 2002). Toll receptors recognize different patterns specific for families of pathogens such as bacteria cell wall, CpG motifs associated with bacterial DNA and double-stranded RNA associated with RNA viruses and transposons. Dendritic cells sense danger and infection indirectly through inflammatory mediators such as TNF-alpha, IL-1β, or PGE-2, whose secretion is triggered by pathogens. Dendritic cells may also be activated by direct binding of receptors such as CD40 (by T cell CD 40L, or apoptotic bodies), FasL and OX40 or receptors for immune complexes. Agents besides receptors are also known, including adjuvant compounds released by cellular injury for the initiation of dendritic cells. Various heat shock proteins are also believed to bind dendritic cells and cause activation. Examples of materials and methods for using danger signals, adjuvants, and maturation factors, are provided in Example 1, wherein nanoparticles were made with hyaluronic acid and/or nickel, which are adjuvants.

Nanoparticles are useful research tools for the study of APCs. Nanoparticles may be made with ligands that bind to receptors on an APC so that the nanoparticle is targeted to the APC and taken up therein. The nanoparticle is degraded in the cell and releases an agent, e.g., a bioactive agent, a visualization agent, a diagnostic agent, or agents that perform some or all of these functions. For example, a nanoparticle targeted to an APC, e.g., a dendritic cell, may contain a marker protein or a plasmid that produces a marker protein that fluoresces. Thus, dendritic cells in a culture, e.g., a skin tissue culture, may be labeled and visualized. For example, an antigenic protein may be delivered, and later used as a marker by detecting it with fluorescently-labeled antibodies that binds in cell culture, tissue culture, frozen section, histology sections, and the like. Example 1 includes nanoparticles made with E-selectin for targeting APCs and plasmid for green fluorescent protein for visualization; as a result, dendritic cells were selectively labeled and visualized.

Further, nanoparticles may be used to selectively deliver agents to APCs for research uses, e.g., for visualization, or to study functional aspects of agents. For example, nanoparticles targeted to dendritic cells may be loaded with nucleic acids. The nucleic acids may encode proteins that are to be expressed intracellularly to determine how the proteins function in the cell. Or, antisense molecules may be delivered to the cell that interferes with a function of genes of interest in the cell. Example 1 describes materials and methods for using nanoparticles to deliver plasmids specifically to APCs. The plasmid of Example 1 was green fluorescent protein, which was used for visualization purposes.

Nanoparticles may also be made with adjuvants, danger signals, adjuvants, and/or maturation factors for use as research tools. The nanoparticles may be added to a cell or tissue culture so as to simulate the presence of a danger signal, adjuvant, and/or maturation factor. The nanoparticles may, for example, be taken up by an APC and elicit an observable response from the APC. The behavior of cells that have taken up particles may be compared to that of cells that have not contacted, or taken up, the particles. Thus activated cells may be compared to unactivated, or lesser activated cells.

Nanoparticles may also be used to make vaccines. Vaccines may be made by creating a nanoparticle that comprises an antigen. The nanoparticle may also include ligands that are specific for APCs. Adjuvants, danger signals, and maturation agents may also be included.

Example 1 describes in a nanoparticle vaccine made using an antigen that was a bacterial DNA, i.e., betagalactosidase, and a dendritic targeting agent, E-selectin. Embodiments wherein the nanoparticle comprised an adjuvant, hyaluronic acid or nickel, are also shown. The nanoparticles were made to be less than about 50 nm in diameter for enhanced intracellular delivery to the nucleus and cytosol of the target antigen-presenting cell, while adjuvant nanoparticles are designed to be greater than about 50 nm to enhance delivery of adjuvant into the clathrin-coated pits for appropriate stimulation of cytokine and inflammatory cascades.

Cancer Treatments

Precancerous cells are routinely destroyed by the immune system. Cancer cells, however, are cells that evade the immune system. Much interest exists in using both non-specific and tumor-specific immunostimulation strategies to increase host immunosurveillance in cancer for the treatment of both primary and residual disease, with the result that the body is trained to recognize its cancerous cells.

Strategies that are being explored for enhancing APC recognition of tumors include ex vivo loading of dendritic cells with tumor antigen peptides by electronic pulsing, or ex vivo transduction of dendritic cells with sequences encoding tumor antigens and reinoculation into the host. Numerous Phase 1 clinical trials have established the feasibility of this approach for treating numerous human cancers. (Lipscomb et. al (2001), Physiol. Rev. 82:97-130).

Works by other investigators have explored more non-specific effects of immunostimulation in enhancing survival from cancer. Multiple administration of plasmid DNA: liposomal complexes (4×100 mcg) has been shown to confer a long-term survival benefit and, in some cases, systemic protection against rechallenge by tumor inoculation. Tumors that were inherently more immunogenic were more amenable to treatment and no protection was conferred by injection of plasmid DNA or liposomes alone. Consistent with capacity of plasmid DNA to be recognized by the innate immune system, DNA from a prokaryotic source conferred protection, suggesting the importance of CpG motifs in upregulating the innate immune system and DC activity (Rudginsky et. al (2001), Gene Ther. 4(4):347-355). Thus the inherent potential immunostimulatory activity of genetic constructs is desirable.

A method of treating or studying cancer is to introduce agents into cancer cells that make the cells targets of the immune system. For example, foreign DNA, proteins, or nucleic acids that produce proteins may be introduced into cancer cells, with the result that they become immunogenic. The introduction of adjuvants or danger signals before, after, or at the same time as the introduction of agents that make the cells immunogenic may enhance the resultant immunogenic response. Or danger signals, e.g., danger signal factors that are introduced into or near cancerous cells may be sufficient to train the immune system to recognize the cells. Such agents may be introduced into or near the cell, e.g., with nanoparticles, or into the same region as the treated cells. Such agents may be used alone or in combination.

Another embodiment involves delivering nanoparticles loaded with antigens to APCs, e.g., dendritic cells. Ligands for targeting APCs are described herein, and may be used for targeting. The antigenic materials in the nanoparticles are released into the APCs, where they are processed and presented to other immune system cells so as to trigger an immune response. Alternatively, nanoparticles without ligands for targeting APCs may be delivered to regions of the body where APCs dwell; such nanoparticles are internalized by APCs, e.g., macrophages, and then release their antigens and thereby stimulate the immune system. Adjuvants and/or danger signals may be used in conjunction with nanoparticles. For example, nanoparticles that comprise adjuvants and/or danger signals maybe made and introduced into the same cells, near the same cells, or in the same region as nanoparticles that have antigenic agents by using an appropriate targeting agent with the nanoparticles. Alternatively, adjuvants and/or danger signals may be introduced into or near a cell, or into a region, without the use of a nanoparticle, e.g., by injection or other introduction. The presence of adjuvants and/or danger signals near APCs may help to stimulate the immune system and train the immune system to respond to antigen associated with nanoparticles.

Cancer may also be treated or studied by introducing nanoparticles comprising adjuvants and/or danger signals into or near cancerous cells, or in the region of cancerous cells. The danger signals and/or adjuvants stimulate the immune system to recognize antigens associated with the cancer cells.

Toxicity and Inflammation

A device that stimulates the immune system preferably avoids toxic cellular effects. Avoiding toxicity in the context of immunostimulation is made more difficult because stimulation of the immune system often involves inflammatory pathways. Further, some studies have implicated the size of particles introduced into the body as being a factor that contributes to inflammation and toxicity. Example 2 shows that nanoparticles prepared as described herein have low toxicity, and are much less toxic than conventionally used particles, e.g., liposomes.

In contrast with the approaches herein, conventional colloidal therapeutic delivery systems, including those under investigation and in use, generally consist of i) species larger than 200 nm in diameter, e.g. microspheres, microparticles or coated organic crystals, and ii) particles in the 70-200 range, e.g. lipid vesicles\liposomes, polymeric conjugates and self-assembling polymeric mixtures reviewed in Brigger et. al, (2002), Adv. Drug Deliv. Rev. 54: 631-651; vol. 47 (2001) of Advanced Drug Delivery Reviews). These systems are characterized by their tendency to be taken up by macrophages of the spleen and liver, also referred to as the reticuloendothelial system (RES). This uptake is mediated by particle binding to serum opsonin proteins (Kreuter et. al (1996), J. Anat. 189:503-505). There is apparently a strong relationship between particle size and RES uptake. Abra et. al showed, using liposomes of decreasing size (460 nm, 160 nm, 58 nm) loaded with radioactive inulin, that 60 nm liposomes did not accumulate in the spleen with increasing dose, while larger ones did. Liver accumulation increased with dose for all sizes of liposomes, but the absolute value of that accumulation was 50% less for 60 nm vs. 460 and 160 nm. Unfortunately, the desirable decrease in RES uptake of the small liposomes was offset by a decrease in stability and tendency to aggregate (Abra et. al. (1981), Biochem. Biophys. Acta 666:493-503). This relationship between particle size and uptake by the major organs of the immune system can also be observed in the problem of aggregate levels in formulations of therapeutic proteins. Here, variation in levels of dimers, trimers, etc. correlates with antigenicity of various IFN-alpha formulas in mice and humans (Braun et. al (1997), Pharm. Res. 14(10):1472-78).

The use of polyethylene glycol-modified lipids to manufacture long-circulating liposomes, e.g. stealth liposomes, has provided a significant advance in colloidal delivery systems by enabling accumulation of such liposomes in certain extravascular compartments, such as tumors (Chonn et. al, (1998) Adv. Drug Deliv. Reveiws 30:73-83). The proposed mechanism for stabilization involves the creation by the lipid conjugates of hydrophillic brush coat around the vesicle that inhibits serum protein binding, complement activation and lipid membrane stabilization. Excessive stability creates different side effect profile. Undesirable uptake of particles by the RES can have unintended consequences in terms of liver macrophage toxicity and a resulting decrease in capacity for clearance of bacteria from the blood. This toxicity was managed by dosing schedule (Storm et. al (1998), Clin. Can. Res. 3: 111-15). Enhanced steric stabilization of liposomes has also been problematic in terms of development of liposomal systems for in vivo gene delivery as controlled destabilization/fusion with the endosomal membrane, a key element in successful delivery of the genetic construct to its site of action in the nucleus or cytoplasm, is intrinsically hindered (Chonn et. al, (1998) Adv. Drug Deliv. Reveiws 30:73-83).

Liposomes, however, are toxic in sufficient concentrations. Thus delivery of a desirable dose of an agent in a liposome may be challenging. Using different colloidal formulations, several groups have identified a specific toxicity syndrome across multiple strains of rodents that is uniquely associated with intravenous dosing of lipid complexes of plasmid DNA (thoroughly described in Tousignant et. al (2000), Hu. Gene Ther. 11: 2493-2513).

This toxicity syndrome most closely resembled endotoxemia, a condition where activated Kupffer cells (activated by complex uptake and recognition of bacterial DNA patterns), damage nearby hepatocytes by generation of inflammatory cytokines and was characterized in rodents by malaise, focal liver necrosis and transient elevation of serum liver transaminases (ALT, AST). Peak levels of serum transaminases correlated with the severity of the syndrome. Effects of the syndrome (mortality) begin at doses >4 mg of plasmid DNA/kg of body weight. Involvement of the innate immune system in this syndrome was indicated by a sustained 10-fold elevation in the DC-associated cytokine IL-12 at 24 hours posttreatment. Other features of this toxicity syndrome included transient cytokine elevation, thrombocytopenia, lymphopenia and biphasic neutrophil changes. As described, DNA-binding receptors have been shown on the surface of both leucocytes and platelets, inducing platelet activation and leukocyte-endothelial binding and subsequent exit from the bloodstream. For lipid complexes, neutrophil losses are apparent only at low doses. Consistent with other models of endotoxic sepsis, a compensatory, dose-dependent neutrophilia obscures this loss at higher doses of lipid plasmid DNA complexes.

Administration of inflammatory DNA complexes upon a background of inflammatory disease synergistically increases these toxicities. In a rodent model of acute inflammatory disease by experimental pancreatitis, administration of 100 μg (4 mg/kg) of lipid complexes doubled 6 day mortality from 40% to 80% in a study using 100 endotoxin resistant mice. Endotoxin-resistant mice were used to rule out effects from endotoxin contamination in plasmid DNA preparations (Normal et. al (2000), Gene Ther. 7:1425-1430).

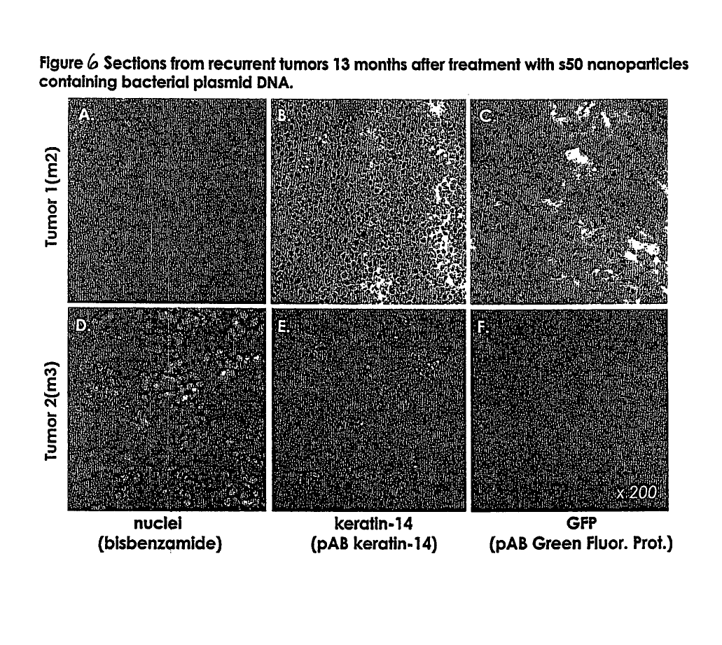

Such a model is used in Example 2, and shows that nanoparticles prepared as described herein are much less toxic than liposomes. In Example 2, nanoparticles were made that comprised plasmid DNA (green fluorescent protein), hydrophobic surfactant of HLB less than 6.0, and tenascin. The tenascin was present and either a high or a low concentration. A high concentration of tenascin is believed to have reduced or eliminated cellular contact with the plasmid DNA, and such nanoparticles are believed to have had a nanocapsulor morphology. A low concentration of tenascin is believed to allow for more contact between the plasmid DNA and cells, at least during the time before the nanoparticle is taken up into the interior of the cell. The nanoparticles ranged in size from about 20-70 nm, except for treatment group 2, which comprised aggregated nanoparticles that collectively had a diameter of approximately 100 nm. FIG. 6 and Table 2 show the treatments that were used: group 1 was a saline only treatment; group 2 was aggregated nanoparticles with a high tenascin concentration and an antisense sequence against CK2alpha (see CK2alpha phosphodiester backbone antisense sequence in Unger, U.S. Patent Ser. No. 60/428,296, filed Nov. 22, 2002); group 3 contained nanoparticles having both tenascin and trehalose but no DNA; group 4 contained nanoparticles having plasmid DNA and a high tenascin concentration; and group 5 contained nanoparticles having plasmid DNA and a low tenascin formulation.

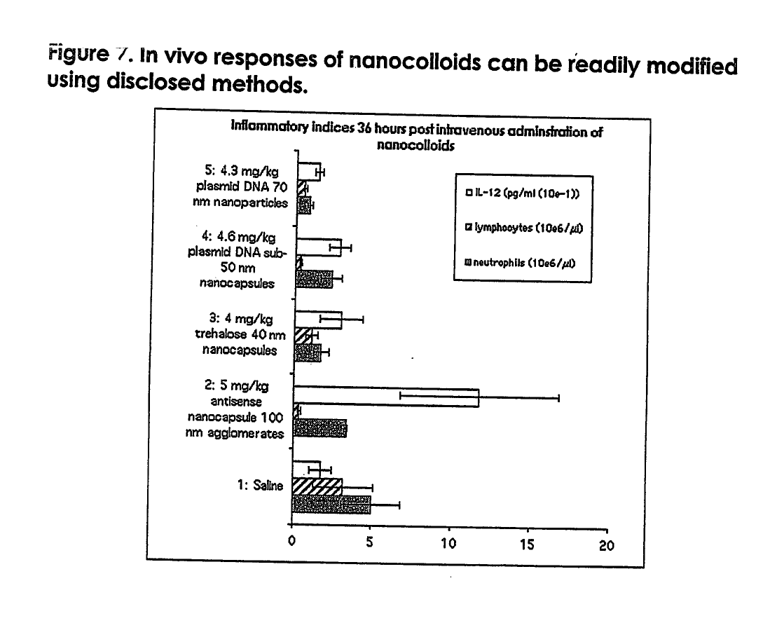

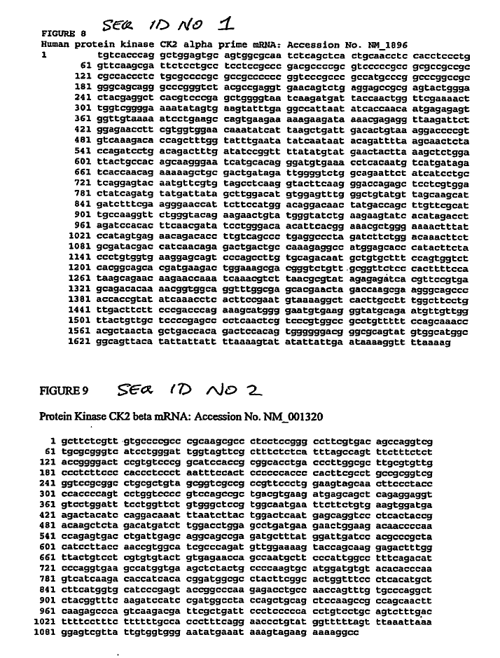

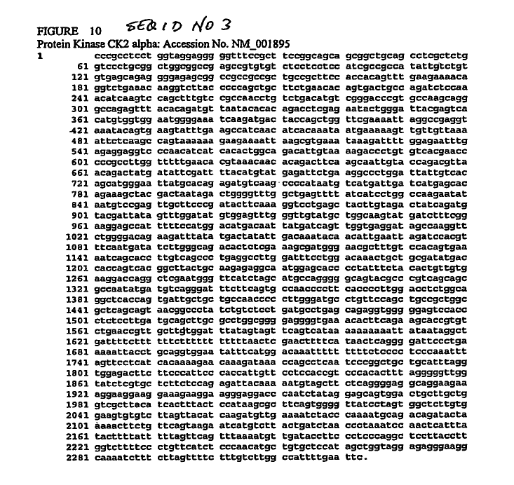

FIG. 7, a bar chart, showed that an inflammatory response like that previously observed in the scientific literature was obtained for Group 2, in terms of cytokine elevation, which was the group that had the large diameter aggregation of particles. The size of the aggregate in Group 2 was similar to the size of some of the particles tested in the scientific literature. Group 5, which was made with a relatively low tenascin concentration, showed neutropenia. In addition, all four mice from Group 5 had full gallbladders, indicating that none of the mice were healthy enough to feed normally.

### EXAMPLES

The following Examples which are intended as illustrations only since numerous modifications and variations within the scope of the present invention will be apparent to those skilled in the art after reading this disclosure.

Reagents:

A. Nucleic Acid Condensing Agents

Poly(ethylenimine) (PEI) at 27 KiloDalton (kD). PEI was used at optimized conditions (90% charge neutralization)

Polyarginine (parg) at 15K molecular weight.

B. Surfactants

2, 4, 7, 9-tetramethyl-5-decyn-4,7-diol (TM-diol): HLB=4-5.

C. Polymers

Recombinant E-selectin or CD62E, 58,800 daltons. Recombinant form consisting of 535 amino acids minus the transmembrane and cytoplasmic domains. This glycoprotein is transiently expressed on vascular endothelial surfaces where it is able to bind ligands on leucocytes. Hyaluronan, recombinant, 1 million kiloDalton (MM kD); Povidone (polyvinylpyrrolidone, PVP) 10,000 kD M; Tenascin, 220 kD; recombinant Granulocyte Macrophage Colony Stimulating Factor (GMCSF) and not bioconjugated.

D. Expression Vectors

pcDNA/His/LacZ, produces betagalactosidase via a CMV promoter, based on pcDNA3.1(Invitrogen), 8.6 kB; pEGFP-c3/p57(Kpn/Sma) Clontech enhanced GFP (green fluorescent protein) expression vector modified with a nuclear localization tag from a cyclin dependent kinase to improve microscopy, 4.6 kB.

E. Detection Agents

Colloidal gold suspension, 100 mg/ml, nominal diameter by light scattering 20 nm (E-Y Industries); Fluorescein-tuned, ZnS outer-shelled, CdSe semiconductor nanocrystals, nominal diameter 10 nm (Evident Technologies), in ethanol, also referred to as quantum dots.

### Examples

### Example 1

### Targeting of APCS and Use of Adjuvants and Danger Signals to Enhance Immunogenicity

Various molecules are suitable for targeting an APC; this Example shows the use of E-selectin and hyaluronan plus adjuvant. E-selectin nanoparticles were tested for functional uptake by potential APCs using an organ-cultured porcine skin model of 8 mm² punch biopsies cultured on an 80 mesh stainless steel grid at an air-media interface using commercially available organ culture dishes, see also Unger et al., 2003. The effect of adjuvants and danger signals was also assessed. This Example shows the use of the adjuvant hyaluronic acid (HA), which was incorporated into a nanoparticle as a polymer associated with the hydrophobic surfactant in the particle. The adjuvant nickel was also used; it was introduced into that particles by using it in solution in a precipitation step in nanoparticle formation, whereby it became incorporated into the particles.

Nanoparticles for immunostimulation studies were manufactured via “dispersion atomization” as previously described using a 8.3 kp plasmid expressing β-galactosidase (βgal, 4112b). Briefly, sub-50 nm diameter nanoparticles (s50-nanocapsules) were produced by: a) dispersing 100 μg of plasmid complexed with 20 μg of 15 Kd polyarginine into sterile water using a water-insoluble surfactant system of 6.5 μg of TM-diol in 50% DMSO; b) emulsifying the dispersed nucleic acid by sonication with a water-miscible solvent, 170 μl of DMSO; c) inverting emulsion with 780 μl of PBS addition; d) coating hydrophobic micelles with: a high concentration of hyaluronic acid (HA) (5 mcg), a low concentration of HA (0.25 mcg), or 5 μg of 58,800 MW rE-selectin plus 50 nm sheep IgG; and e) atomizing ligand-stabilized micelles into a salt receiving solution (200 mM Li⁺, 10 mM Ca²⁺ and optionally 25 ppm amount of Nickel). Following overnight incubation, particles were collected by centrifugation from the mother liquor by decanting and 0.2 μM filter sterilization. Encapsulation yield was measured at 65% using a standard overnight protein K digestion at 56° C. followed by isobutanol extraction and recovery of DNA on an anionic column. Average particle size of the major species was less than 50 nm as measured by tapping mode atomic force microscopy of a 0.1 μg/ml sample dried down on a mica sheet.

The role of adjuvant and/or danger signal addition in nanocapsule immunostimulation was tested by preparing particles that incompletely encapsulate bacterially-derived plasmid DNA (nanoparticles) and the addition of 25 ppm NiCl₂ to the salt receiving solution. Plasmid DNA nanoparticles were prepared out of 1 MM molecular weight hyaluronan as described above using a 4.6 kB reporter plasmid for Green fluorescent protein (GFP) by reducing the addition of polymer from 5 mcg to 0.25 micrograms. Nickel-modified nanocapsules comprised of hyaluronan and pDNA for LacZ were prepared as described with the addition of 0.625 mg to 25 ml of receiving solution. DC's play a well-known role in the initiation of response against simple chemicals as observed to contact hypersensitivity (Manome et. al (1998), Immunol. 98:481-490). Nickel is a well-known contact allergen with a long history of high levels of human exposure through study of corrosion products from implantable prosthetic devices (Hunt et. al, (1992), J. Biomed. Mat. Res. 26: 819-828).

To initiate a punch biopsy test, nanocapsules containing LacZ reporter plasmid were topically applied to 8 mm² biopsies, cultured for 5 days then snapfrozen for cryosectioning and detection of β-galactosidase expression by immunofluorescence microscopy. Cell locations were identified by bisbenzamide counterstain for viable nuclei. Beta-galactosidase was detected for by both a polyclonal antibody against betagalactosidase and a monoclonal antibody against a tag engineered onto the reporter (X-press, Invitrogen). The marker s-100 calcium-binding protein antigen was used and observed to mark cells that were activated, as opposed to unactivated. (Neomarkers Rb-9018). APCs were identified by a monoclonal antibody MSA-3 to porcine MHC II. Mature, activated dendritic cells were detected using monoclonal antibodies against either Cd1a, Cd1c or Dec-205/Cd 205 (Larregina et. al (1997), Immunol. 91:303-13; Inaba et. al (1995), Cell Immunol. 163:148-156). The following table summarizes results from several immunofluorescence analyses.

TABLE 1
Tissue distribution of reporter gene expression and mature dendritic cells in skin biopsy explants
treated topically with various nanocolloid compositions after 5 days of organ culture.

| Nanoparticle Design | | | | | |
|---|---|---|---|---|---|
| (HA in high | | | | | |
| concentration unless | | | | Biopsy distribution of reporter | Distribution of mature dendritic |
| otherwise indicated) | | Dose | | gene expression | cell immunosignal |
| | Plasmid and E-selectin | | 2 μg | Reporter in dendritic cells, | None: dendritic cells not |
| | nanoparticles | | | characterized as long, thin | activated. |
| | | | | fibrous cells; were adhered to | |
| | | | | collagen tendrils | |
| (1) | Plasmid, and E- | (1) | 2 μg + | Reporter in dendritic cells in | Limited punctate clusters of |
| | selectin nanoparticles | (2) | 2 μg | epidermis, had scattered | signal in activated dendritic cells |
| (2) | Plasmid, and HA | | | punctate expression; signal also | in epidermis; limited signal for |
| | in low concentration | | | in MHC II-(+) microvascular | activated dendritic cells (Cd1c- |
| | nanoparticles | | | endothelial cells | (+)) in dermis. |
| | Plasmid and HA | | 2 μg | Reporter in Keratinocytes, | Not assayed. |
| | nanoparticles | | | microvascular endothelial cells. | |
| (1) | Plasmid, and HA | (1) | 2 μg + | Reporter in dendritic cells in | Signal (Cd1c-(+)) showed |
| | nanoparticles | (2) | 2 μg | epidermis; very limited | activated dendritic cells in |
| (2) | Plasmid, and HA | | | reporter expression visible in | epidermis and dermis, with signal |
| | in low concentration | | | MHC-II endothelial cells | in punctate clusters in epidermis |
| | nanoparticles | | | | and dermis. Strong signal |
| | | | | | (Cd205-(+)) also in dendritic cells |
| | | | | | in dermis with veiled, long, thin, |
| | | | | | fibrous morphology |
| | Plasmid, HA, and | | 2 μg | High reporter signal in | Strong signal (cd1c) showed |
| | Nickel nanoparticles | | | punctate clusters of dendritic | strong dendritic cell activation in |
| | | | | cells throughout epidermis and | punctate clusters of cells in |
| | | | | dermis | dermis and epidermis, and |
| | | | | | positive signal (Cd 205-(+)) in |
| | | | | | both dermis and epidermis for |
| | | | | | activated dendritic cells having |
| | | | | | veiled, long, thin, fibrous |
| | | | | | morphology |
| | Nanoparticles with | | 2 μg | Smooth muscle cells, some | No activated dendritic cells (i.e., |
| | Polyvinylpyrrilidone | | | fibroblasts | no Cd205-+ cells present). |
| | substituted for HA | | | | |

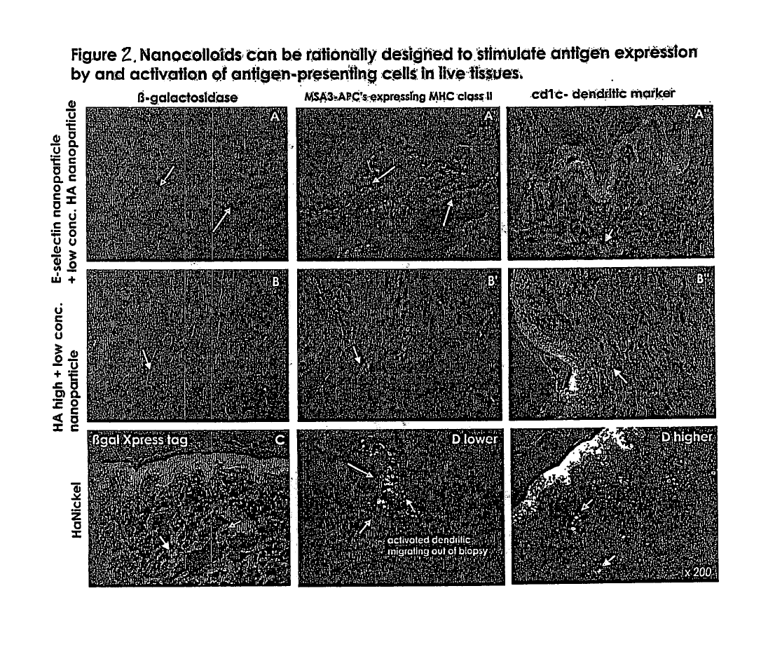

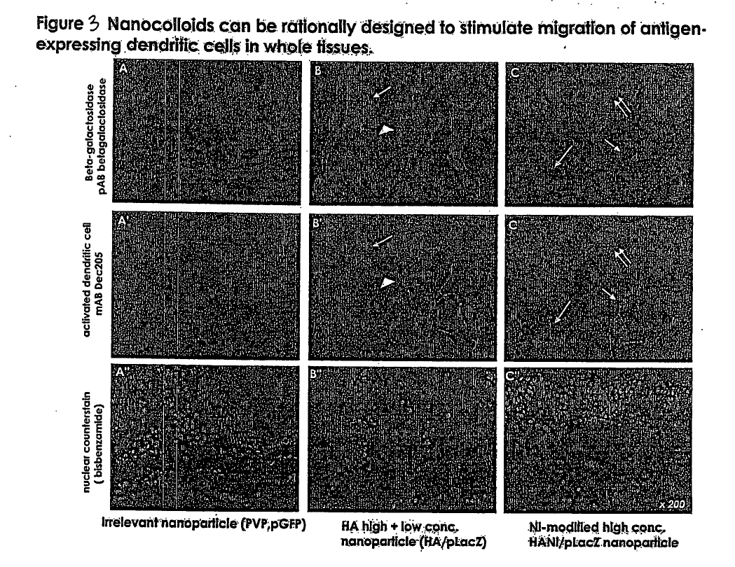

These observations are illustrated in FIGS. 2 and 3. FIG. 2 shows that the E-selectin nanocapsule in combination with the adjuvant HA nanoparticle transduces β-galactosidase expression in a majority of Class-II positive microvascular structures (2A vs. 2A′). Expression of β-galactosidase is also apparent in fibrous cells in view 2A, while expression of Cdc1a, a marker of DC maturation and activation is apparent in view 2A″. Cdc1a immunosignal is visible in the epidermis and in discrete clusters in the dermis. Microscopy for HA nanocapsules combined with HA nanoparticles showed similar results (2B, 2B′, 2B″) with the following differences; i) only a few Class-II microvascular structures showed β-galactosidase expression and ii) more fibrous, β-galactosidase-(+) cells were visible. Levels of Cdc1a in the epidermis and dermal clusters appeared similar (2A″ vs. 2B″). Application of HaNi nanocapsules, however, to punch biopsies resulted in evidence of significant DC transduction and activation 5 days later in sections. Cdc1a levels in epidermis and dermal clusters were very bright and clusters of Cdc1a-(+) were detectable in the very lowest portions of the section towards the subcutaneous fat layer (2D higher vs. 2D lower). β-galactosidase expression was high in HaNi-treated sections in both epidermis and cellular clusters throughout the epidermis.

In FIG. 3, using frozen sections from similar experiments, the identification of cells were examined more closely in the dermal clusters observed in FIG. 2 using the monoclonal antibody specific for dendritic cells Dec-205 (Inaba et. al, (1995). In punch biopsies treated with PVP nanocapsules containing a reported gene for GFP, GFP expression was observed in smooth muscle cells as expected but no epidermal expression of β-galactosidase or Dec-205 indicating that PVP nanocapsules do not deliver their cargo to immune cells nor do they activate skin dendritic cells (3A, 3A′, 3A″). In contrast and consistent with results presented in FIG. 2, HA nanocapsules in combination with adjuvant HA nanoparticles transduce β-galactosidase expression in epidermis and fibrous cells (3B, narrow and broad arrows). View 3B′ identifies these fibrous cells as Dec-205 positive and thus DC. Again, as in FIG. 2, application of HANi nanocapsules containing the LacZ reporter induce higher levels of β-galactosidase expression in epidermis and dermal clusters. View 2C′ identifies these cell clusters as being Dec-205 positive and thus clusters of activated, migrating DC. The increased levels of β-galactosidase expression in the epidermis observed in HANi treated punch biopsies is consistent with the location of Langerhans cells suggesting that HANi particles may significantly stimulate both LC and DDC (double arrows, 3C vs. 3C′). As previously published, s50 nanoparticles containing β-galactosidase without any form of adjuvant (e.g. exposed Cpg's, nickel) transduce only basement membrane keratinocytes and microvascular endothelial cells in porcine skin biopsies (PCT WO 00164164A2).

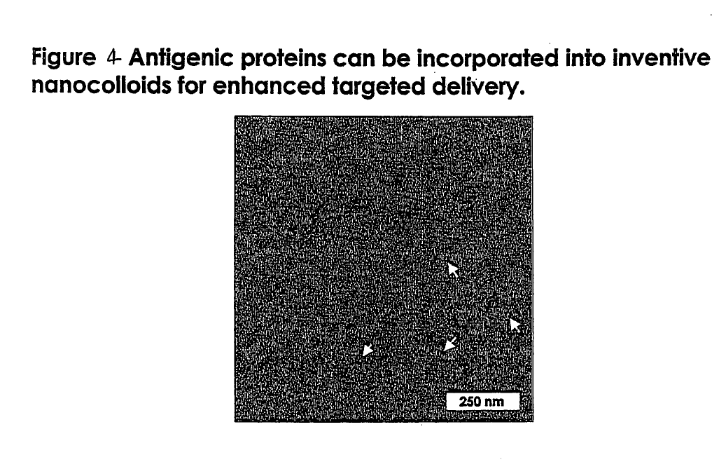

Taken together, these results indicate that adjuvant-modified, nanoparticles described herein, can significantly activate key effectors of immune response in tissue from a large mammal. Various strategies for enhancing immune response from DNA vaccines have been reported in the literature including boost with a protein antigen, coadministration of cytokines and various adjuvants including viral vectors and alternate routes of administration. (Weeratna et. al (2000)). FIG. 4 shows successful incorporation of β-galactosidase protein (42 Kd) into s50 nanoparticles of HANi. The average dry diameter of these particles was 15±3 nm by atomic force microscopy (mean±SD). Certain strategies known in the art for enhancement of immunomodulatory activity may be used in combination with nanoparticles described herein (e.g., U.S. Pat. Nos. 5,589,466, 5,723,335, 6,303,114, 6,404,705, 6,413,942, 6,475,995, and PCT WO 00182964 and Johansen et. al, (2000)).

### Example 2

### Demonstration of Immunostimulation by Nanoparticle Dosage Form In Vivo

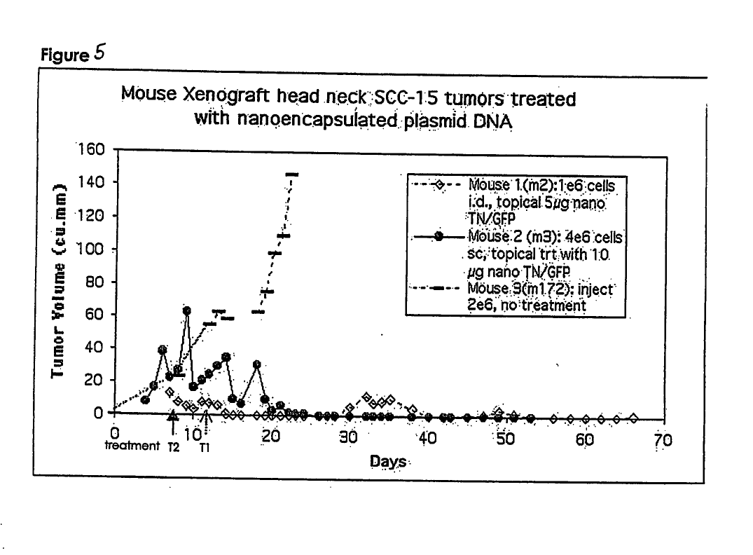

We have previously shown that nanocapsules comprised of tenascin, an extracellular matrix protein produced by tumor cells, selectively delivery genetic constructs and other cargo into in vitro tumor nests in organ culture by topical application, see commonly owned and assigned U.S. patent application Ser. No. 09/796,575 filed Feb. 28, 2001 and Ser. No. 10/378,044, filed Feb. 28, 2003, and also Unger et. al, (2002) AACR Proceedings, 43: 577. In organ-cultured tumor nests, carcinoma cells selectively and uniformly express the Green Fluorescent reporter gene. In a nude xenograft mouse study of human head neck cancer employing the squamous cell carcinoma line SCC-15, two mice were treated topically at the tumor site with 5 and 10 microgram doses of tenascin encapsulated bacterially-derived plasmid DNA. Tumors were palpable with starting diameters of 3 to 4 mm and were initiated on the right, upper mid flank. It was observed that cycling of the tumor size from palpable to visible which eventually regressed completely. A timecourse of tumor volume is illustrated in FIG. 5. Notice that more rapid control of tumor growth is achieved in the mouse treated with 10 rather than 5 μg of nanoencapsulated plasmid, despite the fact that the initial tumor inoculation was larger in the mouse treated with 10 μg suggesting a dose response between nanocapsule dose and onset of tumor growth control. (1e6, 5 μg vs. 4e6, 10 μg). An untreated SCC-15-xenograft mouse inoculated with 2e6 tumor cells is included for comparison to show tumors normally grow steadily. Tumor control continued for 12 months in both mice, at which point, tumors appeared and began to grow. These new tumors were located in different locations than the original primary tumors. Tumors were recovered upon sacrifice and assayed for the presence of keratin-14, a marker of human head neck carcinoma, to determine whether tumors were derived from the original tumor inoculation. Results from this analysis are illustrated in FIG. 6. The new tumor in the mouse treated with the higher dose of s50 nanocapsule was located low in the abdominal area (mouse 3) was not keratin-14 or GFP positive indicating it was a spontaneous tumor of murine origin (5D-F). The new tumor in the mouse treated with the lower dose of s50 nanoparticles (mouse 2) was located near the inguinal lymph node. Upon necropsy, this mouse had significant tumor burden in its spleen. This new primary tumor was both keratin-14 and GFP positive (5A-C) suggesting that GFP s50 nanocapsules had successfully transduced tumor cells in the original tumor but had not elicited a strong enough innate immune response to successfully eradicate the tumor. Foreign antigen genes transfected into tumor cells are known to behave like tumor antigen (Condon et al. (1996)). Antigen-presenting cells are also known to scavenge apoptotic bodies and in this way acquire antigen (Albert et. al, J. Exp. Med. 188:1359-68; Sasaki et. al (2001), Nature Biotech 19: 543-47). The confluence of two inflammatory stimulators, one the apoptotic milieu of a tumor, the other, bacterially-derived plasmid DNA can combine to provide anti-tumor effects. This example illustrates how targeted delivery of an inflammatory species (e.g., antigen or adjuvant) can function to localize and modulate inflammatory activity for therapeutic benefit.

These results suggest that ultra-small particles of the invention are, by themselves, not immunostimulatory per se, but when loaded with i) antigen and adjuvant or ii) antigen in presence of immunostimulatory environment, they become efficient effectors of immunomodulation.

### Example 3

### Manipulation of Standard Nanocolloid Dosage Form to Modulate Immunostimulation In Vivo

A series of particles were prepared that comprised the extracellular matrix protein tenascin and a plasmid DNA reporter construct for DNA. Tenascin can be used to specifically target particle for intracellular uptake by solid tumors (Unger et. al, (2002) AACR Proceedings, 43: 577). Particles were manufactured as described in Example 1 with some exceptions to vary both size and encapsulation state. Encapsulation state was varied in the nanoparticle formulation by reducing the tenascin component from 2.5 μg to 0.25 μg. Size was varied in the antisense DNA formulation by using an excess (50 μg) of 15,000 MW polyarginine as a condensing agent to create aggregates rather than single particles.

Non tumor-bearing nude mice (˜1-1.5 years old, 3-4 per group), were administered 100 μg doses by tail vein injection and sacrificed 36 hours later. Whole blood and serum were collected for hematological analyses, IL-12 quantitation and major organs were dissected out, weighed and collected. Livers were read by a board-certified veterinary pathologist. The study was designed to mimic as much as possible studies performed in Tousignant et. al (see above). WBC counts and IL-12 levels are shown in FIG. 3; serum chemistry profiles and tissue data are summarized in Table 2. A gall bladder state of empty indicates that the mouse had been eating recently and thus was feeling well at some macro level.

TABLE 2
Acute liver, kidney profile after 36 hours of exposure from mice treated
intravenously with single doses of various ultra-small particles.

| | | | | | | | | Liver |
|---|---|---|---|---|---|---|---|---|
| | Dose | | ALT | AST | BUN | Albumin | Gall | histo- |
| Treatment | (mg/kg) | Size | (U/L) | (U/L) | (mg/dl) | (g/dl) | Bladder | pathology? |
| 1 - saline | 0 | None | 33 ± 5 | 111 ± 19 | 21 ± 6 | 3 ± 0.6 | Empty, | none |
| | | | | | | | 4/4 | |
| 2 - Antisense DNA/ | 5 | 100 nm | ND | ND | 22 ± 3 | 2.3 ± 0.3 | Empty, | some |
| tenascin nanocapsule | | aggregates | | | | | 2/3 | |
| 3 - Trehalose/tenascin | 4 | L.T. 50 nm | 50.3 ± 8 | 232 ± 59 | 24 ± 4 | 1.9 ± 0.1 | Empty, | none |
| nanocapsules | | | | | | | 4/4 | |
| 4 - Plasmid DNA/ | 4.6 | 20 nm, | 65 ± 6 | 311 ± 28 | 17 ± 0.3 | 1.9 ± 0.1 | Empty, | none |
| tenascin nanocapsule | | 40-60 nm | | | | | 4/4 | |
| 5 - Plasmid DNA/ | 4.3 | Toroids, | 45.8 ± 3 | 95 ± 11 | 18 ± 1.4 | 1.7 ± .05 | Full, | none |
| tenascin nanoparticle | | 60-70 nm | | | | | 4/4 | |

* data are reported as means ± standard errors, bolded values are significantly different from saline treatment, p ≦ 0.5, ANOVA/Tukey. ND samples were too hemolyzed for serum chemistry analysis.

Our data indicates that ultra-small particles, even those bearing inflammatory drug loads such as bacterially-derived plasmid DNA, are less inflammatory than corresponding, but larger, particles bearing exposed DNA. No significant organ weight differences were found between experimental groups. In the largest, but fully encapsulated formula (Formula 2), some evidence was observed of liver toxicity concomitant with a 10-fold elevation in IL-12 similar to that observed in Tousignant et. al. Nanocapsules containing the sugar trehalose (Formula 3) were completely unremarkable, other than to note that the decreasing trend in lymphocytes (2-fold) was noticeably different from the non-significant 10-fold decrease in lymphocytes measured in formulations containing antisense and plasmid DNA. The plasmid nanocapsule formula did show a 3-fold elevation in serum AST levels. Consistent with the expected behavior of a controlled release dosage form, the continued circulation of the tumor-targeted nanocapsules in a non-tumor-bearing mouse would lead to a sustained, release of plasmid DNA and lower peak exposure levels. In the Tousignant et. al study, a maximum 8-fold increase in AST occurred 48 hours into the study. Mice treated with very small plasmid DNA nanoparticles showed increased functional evidence of toxicities over nanocapsule in that mice were not eating, albumin levels were reduced and hemograms indicated significant neutropenia from controls. Nanoparticles would be expected to degrade extremely rapidly in serum, contributing to more rapid kinetics in its single dose toxicity profile. Nonetheless, neutropenia, as discussed in the Tousignant et al. study was an indication of toxicity albeit very low levels of toxicity such that compensatory neutrophilia had not been initiated. Ultra-small particles may be made and used to improve toxicities associated with colloidal carrier systems and can be manipulated to exert a range of inflammatory effects as required by the application.

### Example 4

### Nanoparticles Used for Imaging and Biochemical Detection

With the continued improvement in understanding of biological and physical process at the molecular levels, a need continues for agents to affect improved detection and analysis of these processes with respect to sensitivity and resolution. To test whether s50 nanoparticles made with hydrophobic surfactants having an HLB of less than 6 could be used as contrast media for detection of localized cellular events, tumor-targeted nanoparticles were prepared containing electron-dense colloidal gold as the agent in the nanoparticles. These nanoparticles were added to, and detected for, organ culture tumor nests using X-ray fluoroscopy following application of tumor-targeted capsules. X-rays do not pass through electron-dense materials creating a signal which can be detected and processed. Colloidal gold labels have long been used as imaging agents in electron microscopy, x-ray microscopy and other ultramicroscopies (Chapman et al. (1996)).

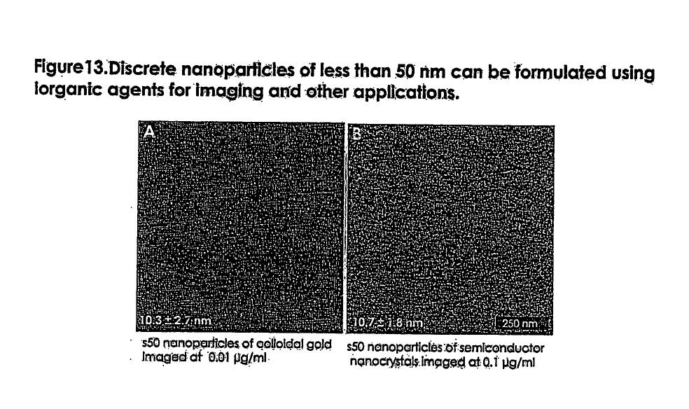

Particles were prepared for the imaging experiment as described in Example 1 with the following changes in proportions; i) 100 μg of nominal 20 nm, 100 mg/ml, colloidal gold dispersion was substituted for the plasmid DNA and ii) 5 μg of 250 KDa tenascin was substituted for recombinant E-selectin. The particle size distribution of final particles were characterized by tapping mode atomic force microscopy (See FIG. 13A). Results showed that recovered tenascin-coated gold nanoparticles were extremely uniform and had a dry average diameter of 10.3±2.7 nm. Tumor-bearing biopsies were prepared by injecting 8 mm punch biopsies of porcine skin, collected aseptically, with varying amounts of the head neck carcinoma cell line, Ca-9-22. Inoculated biopsies were then organ-cultured for 24 hours, before topical application of tenascin nanoparticles containing colloidal gold and further cultured for additional 72 more hours before imaging. Biopsies were imaged on a Siemens Cardioskop-U using an input energy of 8 kV and 25 mA. Position of biopsies with respect to the scanner was adjusted using telephone books and the same wire mesh from the culture apparatus was used in all scans for later image normalization. Image data was collected on videotape, digitized and high-energy (bright; fluoroscopy pulses between dark and bright) images were collected for analysis using the Ifinish software from MediaOne (Marlborough, Mass., USA). Images were processed and analyzed in Adobe Photoshop v. 5.5 by deinterlace filter. Following normalization to the included reference screen, the integrated density of a 963 sq. micron circle was measured using The Image Processing Toolkit within Adobe Photoshop (Reindeer Games Inc, Gainesville, Fla., USA). The following table summarizes the experimental conditions and calculated signal intensity results.

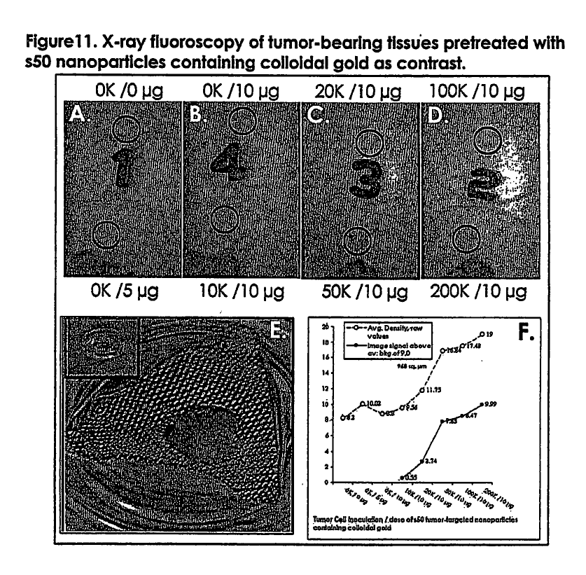

TABLE 3
Results from imaging study of tumor-inoculated tissue.

| | | | Image Signal |
|---|---|---|---|
| Biopsy (position | Tumor | Dose of TN-Gold | (intensity sub |
| in FIG. 11) | Inoculum | s50 nanoparticles | nearby bkg.) |
| 8 (A. top) | none | 0, treated with | 8.2 |
| | | 5 μg of nano- | |
| | | particle containing | |
| | | plasmid DNA | |
| 7 (A. bottom) | none | 5 μg | 10.02 |
| 1 (B. top) | none | 10 μg | 8.8 |
| 2 (B. bottom) | 10,000 | 10 μg | 9.56 |
| 3 (C. top) | 20,000 | 10 μg | 11.75 |
| 4 (C. bottom) | 50,000 | 10 μg | 16.84 |
| 5 (D. top) | 100,000 | 10 μg | 17.48 |
| 6 (D. bottom) | 200,000 | 10 μg | 19 |

Results from this analysis are illustrated in FIG. 11. The top row of the figure (A.-D.) shows the processed fluoroscopy images with a dark circle identifying the analysis area for image intensity; tumor and capsule dose are labeled above each frame. View E and E inset illustrate the porcine biopsy organ culture setup and in view F signal intensity is graphed against increasing tumor dose. The results show that signal intensity (darkness) increases with tumor inoculation dose suggesting that tumor-bearing biopsies either retain more gold particles or accumulate them in point locations to increase signal (scatter). Following subtraction of average background (˜9, average of biopsies 1, 2 &7), this relationship reduces to a linear correlation showing a 10-fold increase in signal intensity for a 20-fold increase in tumor inoculum in a very small area (FIG. 11F). The darkness change was detectable in biopsies inoculated with 50,000 cells or greater by visual observation on a video monitor in the operating room set-up of this experiment.

It was further investigated whether s50 gold particles accumulated in tumor nests by sectioning frozen biopsies and detecting for carcinoma and particle location independently. Tumor nests were identified by comparison of immunofluorescent signal for the integrin and tenascin receptor αᵥβ₆ using a monoclonal antibody (Chemicon) and bisbenzamide counterstain for nuclei position. s50 gold particles were detected using a standard silver enhancement kit for catalytic precipitation of silver nitrate onto the gold according to manufacturer's instructions (Sigma). Briefly, this consisted of incubating sections with a 1:1 volumetric mixture of reagent for 5 minutes, washing slide with water and observing section under a light microscope. Coverslipping is not required.

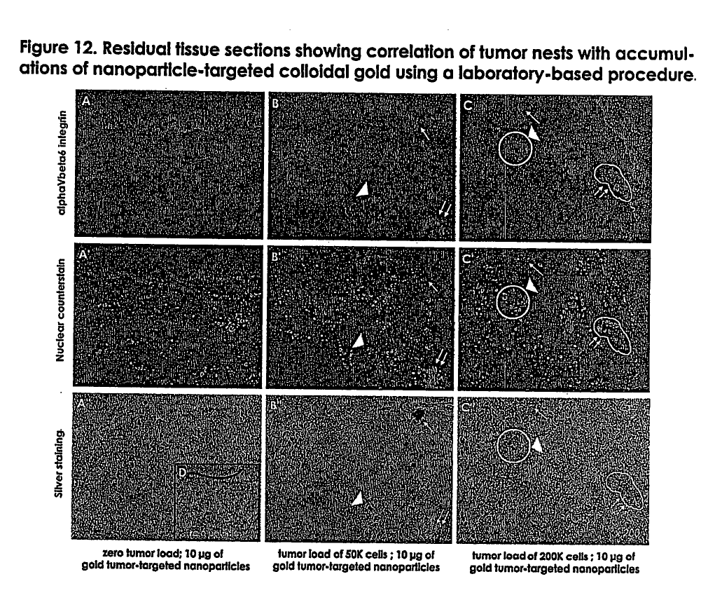

Results of this analysis showing sections from biopsies treated with gold particles but not inoculated with tumor cells (12A-A″), biopsies treated with gold particles and inoculated with 50,000 tumor cells (12B-B″) and biopsies treated with gold particles and inoculated with 200,000 tumor cells (12C-C″) are illustrated in FIG. 12. In column A, it was observed that basal keratinocytes express αᵥβ₆ immunosignal as would be expected for wound-phenotype keratinocytes in an ex-vivo biopsy (12A vs. 12A′). Baseline noise as indicated by deposition of silver composition was extant. However, this background of silver signal was related to the execution method and not co-localization with gold nanoparticles as background appeared similar to sections not treated with gold particles (A″ vs. A″ inset). In addition, these particles appeared to be confined to spaces between intact tissue, suggesting need for improvement in blocking nonspecific adhesion to the slide itself. In FIG. 12, column B, it was observed that, for biopsies treated with 50,000 cells, that an αᵥβ₆ signal was now distributed throughout biopsy indicating the existence of multiple “nests”. Distinct clumps of silver particles (arrows) corresponded spatially with intense areas of αᵥβ₆ signal in the dermis. There were few silver clumps in the interstitial spaces. In column C of FIG. 12, it was observed that for sections from a biopsy treated with colloidal gold and inoculated with 200,000 tumor cells, that αᵥβ₆ signal was now enhanced in the epidermis as well as distributed through out the dermis, indicating that carcinoma cells were colonizing epidermis. Consistent with this, silver deposits now identified the dermal epidermal junction (C″ vs. A″, B″). In general, silver deposits were now located on tissue, compared to control sections and larger areas of silver clumps, that corresponded with more intense tumor staining appeared gray (column C, circles and arrows). Thus s50 nanoparticles are useful for rapid detection and imaging of events at a molecular level cellular level, and tissue level in both a laboratory and intraoperative setting.

### Example 5

### Utility of Inventive Nanocolloids for Imaging and Detection Based on Functional Activity

Nanoparticle uses for imaging and detection were further established by preparing s50 nanoparticles comprised of fluorescent semiconductor nanocrystals, also referred to as quantum dots (qdots, described in U.S. Pat. Nos. 6,301,660, 6,319,426, and 6,326,144, incorporated herein by reference). s50 particles containing hydrophobic surfactant and nanocrystals were prepared for the imaging experiments as described in Example 1 with the following changes in proportions; i) 100 μg of nominal 10 nm, 2 mg/ml, nanocrystal dispersion in ethanol was substituted for the plasmid DNA and ii) 5 μg of polyvinylpyrilidone or 20 kD GMCSF was substituted for recombinant E-selectin. Particle size distribution of final particles were characterized by tapping mode atomic force microscopy (See FIG. 13B). Results showed that recovered PVP-coated quantum dot nanoparticles were extremely uniform and had a dry average diameter of 10.7±2 nm.

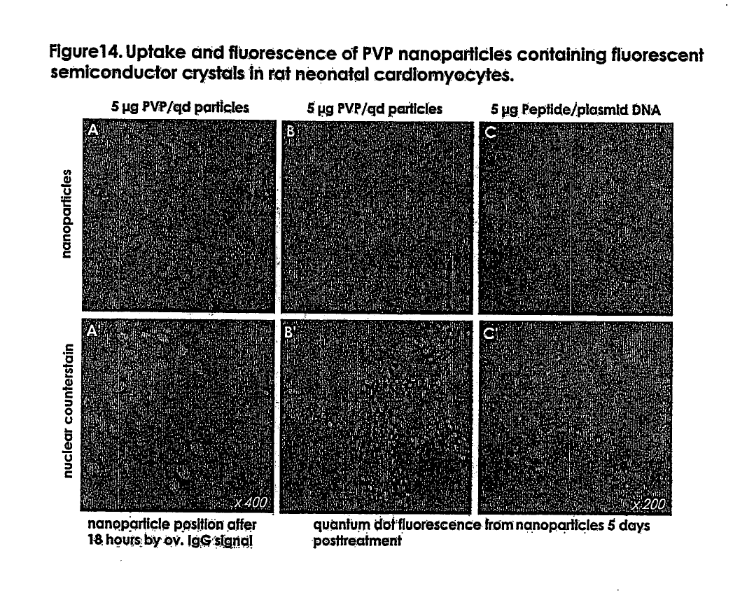

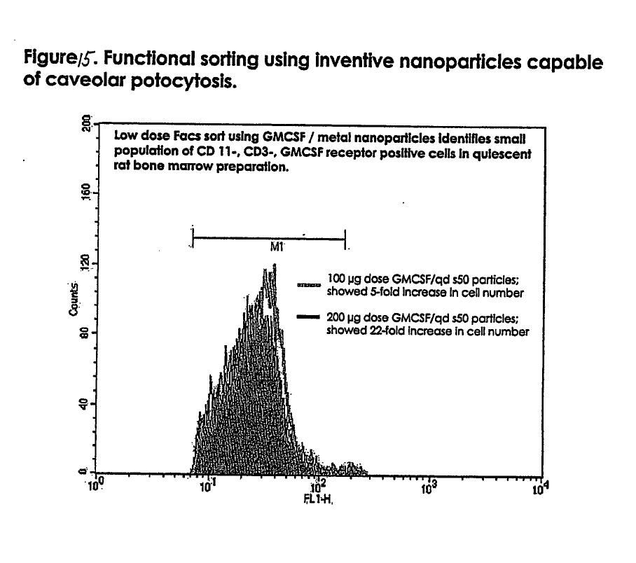

In vivo use of nanocrystals for fluorescent imaging has been complicated by problems with colloidal stability and aggregation (Dubertret et al., (2002), Intracellular uptake of nanocapsule conjugates has been reported but was observed as a punctuate pattern consistent with aggregate uptake by clathrin-coated endosomal vesicles (Akerman et al., (2002)). Five days following application of 5 μg of PVP nanoparticles containing green fluorescent qdots, we observed strong nuclear signal in rat neonatal cardiomyocyte cultures. These results are illustrated in FIG. 14. Nuclear fluorescence continued following cell fixation with 2% paraformaldehyde (B vs. B′). No nuclear fluorescence was observed in cultures treated with nanoparticles containing a plasmid luciferase reporter (C vs. C′) and nuclear uptake of PVP nanoparticles containing nanocrystals was confirmed 18 hours after application by detecting for nanoparticles via immunodetection of ovine IgG “spiked” into the particle coating in combination with anti-sheep antibodies. This experiment illustrates the superior nature of imaging agent delivery by the nanoparticles as described herein.

Because inventive nanocolloids can be optionally prepared of a size to undergo intracellular uptake via caveolae vesicles, the potential implications of combining this functional activity with detection were investigated. Suspensions of bone marrow cells were prepared by flushing media through the femur of a rat, lysing red blood cells, washing and counting cells, then culturing said cells in RPMI media together with 10% fetal calf serum and antibiotics as described in Grauer et. al (2002). Individual cultures were incubated with or without s50 nanoparticles comprised of GMCSF and fluorescein-tuned qdots for 3 days, then counted and analyzed for fluorescence uptake by FACS. Concomitantly, bone marrow cells were analyzed for CD11 (Macrophage/NK cell marker, Serotec) and CD3 (T Cell marker, Serotec) using phycoerythioetin-conjugated antibodies. The experimental design and results for this experiment are summarized in the following table:

TABLE 4
Functional sorting of primary bone marrow
stem cells using s50 qdot fluorescence

| | | | | Average | |
|---|---|---|---|---|---|
| | | Final | | fluorescence | Percent of total |
| Cul- | | Cell | Fold- | for gated | population of |
| ture | Treatment | Count | increase | population | 20,000 events. |
| 1 | 200 μl buffer | 900,000 | 1 | ND | ND |
| 2 | 100 μg | 2e6 | 2 | ND | ND |
| | GMCSF s50 | | | | |
| 3 | No addition | 6e6 | 4 | ND | ND |
| 4 | 200 μl buffer | 800,000 | 1 | 23.95 | 61.67 |
| 5 | 100 μg | 6e6 | 6 | 29.43 | 64.16 |
| | GMCSF s50 | | | | |
| 6 | 200 μg | 22e6 | 22 | 26.35 | 62.92 |
| | GMCSF s50 | | | | |
| Note: | | | | | |
| Cultures 1-2 were derived from one femur and 3-6 from the other. For FACS, excitation filters were set at 488 nm and emission filters at 518 nm on a BD FACSort with CellQuest software. Fluorescent cells were clearly observed in cultures preliminary to preparation for sorting. Cell viability was greater | | | | | |
| # than 95%, by trypan blue exclusion, preliminary to staining for FACS indicating that cells were not fluorescent because of cell death. Note that e6 refers to 10⁶, e.g., 6e6 = 6(10⁶). | | | | | |

These results indicate that the GMCSF, formulated as an inventive nanoparticle coating, was capable of stimulating population expansion compared to buffer alone and thus can be considered biological functional as formulated (cultures 1, 4 vs. 5, 6). This consistent retention of biological activity in the described particles, regardless of capsule material, contrasts strongly with losses of activity frequently encountered during development of targeted agents involving conjugation strategies. Treatment of bone marrow cultures with increased amounts of GMCSF (2-fold) increased growth four-fold (culture 5 vs. 6).

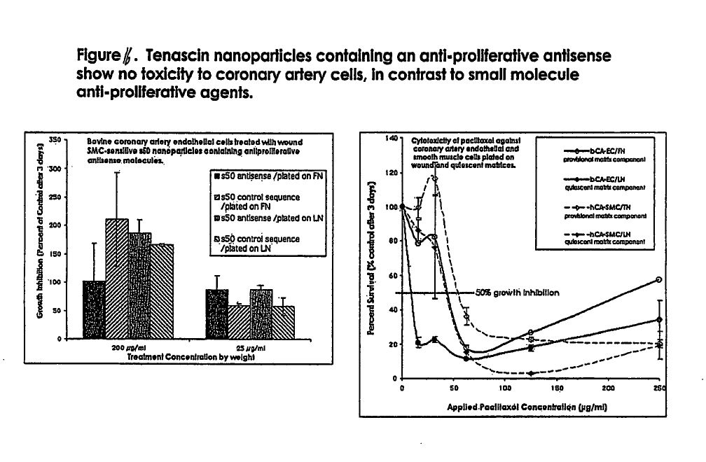

Fluorescence-activated cell sorting (FACS) analysis indicated that at low dose application, approximately 2.5% of cells took up the GMCSF particles compared to 1.25% for the high-dose group. These results suggest that GMSCF stimulation either promoted expansion of a cell population that did not take up capsules or that with the increase in cell numbers (4-fold), the qdot dose (2-fold increase) was no longer sufficient to maintain the same level of signal (signal decreased by 50%). The later interpretation is more likely as examination of the fluorescence profile in FIG. 15 (where cell number is plotted as a function of fluorescence intensity) shows that the peak of the profile shifts to the right, rather than the appearance of second smaller peak upon application of the particles containing the quantum dots. A shift in the peak of the main population indicates that uniform uptake is occurring. In all populations examined, qdot positive cells were not were not CD-11/Mac-1 or CD-3 positive indicating that cells with these surface markers were not present. GMCSF stimulation is expected to increase CD-11 positive cells, however, our cultures were aged for only 3 days, and Mac-1 is not expressed by immature macrophage precursors (Clarke et. al, (1998)).

It may be concluded that s50 nanoparticles could be used advantageously to detect cellular events by combining imaging with functional cellular activity. The heterogeneous nature of the nanoparticles enables simple combination of imaging agents with targeting molecules and biological activity provided by the nanoparticle coating that is readily distributed through tissues as illustrated by the topical application used in Example 4.

### Example 6

### Utilization of S50 Nanoparticles for Cancer Assessment in the Periphery and Transplantation

This prophetic example describes certain diagnostic uses of nanoparticles. s50 nanoparticles for tumor cell detection e.g. metastatic breast cancer with bone marrow infiltration are produced as described in Example 5 containing a quantum dot core with the change that 5 μg of the extracellular protein tenascin is substituted. Alternatively, tenascin could be replaced or combined with thrombospondin, osteopontin, osteonectin or any epithelial cell ligand. A feature of metastatic breast cancer cells is their upregulation of receptors for tenascin prior to their exit from the primary tumor site (Reiss et al., (1997) Breast Can Res. & Treat.)

Because tumor cells express preferentially express receptors for these epithelial-derived tumor cells, quantum dot uptake will occur. Tumor burden can be calculated by:

1. Incubating nanoparticles with bone marrow aspirate or mobilized peripheral blood sample collected from breast cancer patient.

2. After incubation period greater than 4 hours, quantitating epithelial cell (breast cancer cell) uptake by flow cytometry to detect quantum dots fluorescence

3. Calculating tumor cell frequency, i.e. 1 tumor cell per 106 bone marrow cells

s50 nanoparticles for use in treatment of breast cancer by ex vivo clearing of tumor cells from stem cell populations prior to autologous stem cell transplantation may be prepared similarly with the change that 100 μg of a cytotoxic agent are substituted for the quantum dot. Examples are doxorubicin and antisense to the alpha subunit of protein kinase CK2 alpha. In vitro cytotoxicity of these formulations against prostate and head neck carcinoma lines is documented in Unger et. al, (2003) and U.S. patent application Ser. Nos. 09/796,575 and co-pending U.S. patent application Ser. No. 10/378,044. In one embodiment, ex vivo treatment of precursor stem cells includes the following steps: Producing nanoparticles with cytotoxic core and coating for epithelial cell uptake; Incubating nanoparticles with stem cells harvested for autologous transplantation (i.e., bone marrow harvest or mobilized peripheral blood collection); After incubation for more than 4 hours with optional multiple rounds of treatment, washing stem cells using a cell washer to remove targeted tumor cells; Transfusing stem cell transplant or cryopreserving until transfusion is required.

### Example 7

### Utilization of Nanoparticles for Delivery of Therapeutics into Blood Vessel Wall

Much interest has been expressed in gene therapy approaches for alleviation of vascular proliferative and inflammatory dysregulation in atherosclerosis and restenosis (reviewed in Smith et. al, (2001), Feldman et. al, (2000) and approaches to improve the efficiency of gene transfer into the medial and adventitial regions of the artery have been unsuccessful including microneedle strategies. Intramural retention times so far have been too low. The retention times of most locally delivered bioactive agents so far is hours to days with while the greatest decrease in luminal diameter of injured arteries is in the range of weeks to months. In addition, stents coated with nonerodible polymeric matrices can incite inflammatory response and bioactive agent strategies still suffer from the risk of systemic distribution. Finally, current approaches to manipulation of the arterial microenvironment effect all cells in the environment. Brachytherapy or the exposure of wounded artery to irradiation is effective in increasing patient outcomes for up to three years after a stenting procedure. However, brachytherapy, also has significant deleterious effects, including late thrombosis formation at the stent site from failed endothelial regeneration, resulting in significantly higher rates of myocardial infarction. Problems occur when antiplatelet regimens are stopped (Bennet et. al, (2003)) suggesting that late thrombus forms because an anti-thrombotic endothelial layer has not reformed. Recent data from patients, experiencing recurrent instant restenosis, one year after treatment with a paclitaxol derivative-eluting stent indicated continued platelet aggregation due to failed reendothelialization and inflammation from unresorbed polymeric materials (Virmani et. al, (2002)).

In commonly assigned co-pending patents, U.S. patent application Ser. Nos. 09/796,575 and 10/378,044, it is shown that i) s50 nanoparticles comprised of PVP will deliver reported gene uniformly and with high efficiency, across an intact endothelium, to smooth muscle cells of the adventitia in an ex vivo porcine femoral artery preparation, while particles comprised of fibronectin deliver to the medial microvasculature, ii) s50 nanoparticles comprised of tenascin will deliver reporter gene efficiently and specifically into wound-phenotype human coronary artery smooth muscle cells, cultured on proteins deposited from fetal calf serum and artificially wounded with a pipette tip and iii) s50 nanoparticles of hyaluronan, compounded into a binder comprised of solution of mucin and sucrose and dip-coated onto sutures are released from the suture into tissue and taken up by microvascular endothelial cells at least 500 μm from the suture.

Because of the important role, reendothelialization plays in long-term patency of implanted stents, we examined the sensitivity of bovine coronary artery cells to a growth-inhibitory antisense construct formulated into tenascin s50 particles, which are taken up by wound-phenotype smooth muscle cells in a standard MTT assay in a 96 well plate format (Faust et. al, (2000)). This antisense to the alpha subunit of casein kinase 2 in a phosphodiester backbone has shown IC 50's for growth inhibition against chemoresistant carcinoma cell lines ranging from 1 to 20 μM (5 to 125 μg/ml) in vitro (Unger et. al, (2003)). Growth inhibition was compared for bCA-EC's plated on both laminin and fibronectin. Laminin is a major component of normal basement membrane and is known to foster a more quiescent phenotype through adhesion-mediated signaling, while fibronectin is a major component of provisional matrix deposited from serum onto the original fibrin matrix created by aggregating platelets (Bennet et. al (2003)). Microtiter plates were pretreated with extracellular matrix proteins by incubating plates for 4-6 hours at room temperature in 20 μg/ml of laminin (Sigma) or overnight at 37° C. in media containing 20% fetal calf serum. Wells were plated with 7,000 endothelial cells, treated 8 hours later and incubated for 3 days before applying WST reagent to assess viability by enzyme activity.

No growth inhibitory effect was found for antisense tenascin nanoparticles containing either a sense or antisense construct on endothelial cells plated on either substrate. Sense construct were composed of a morpholino format and antisense constructs were composed of either a morpholino or a chimeric phosphodiester 2-o-methyl-modified RNA. Both formulations showed good activity on carcinoma cells. Results are illustrated in the FIG. 16A. A potential overall trend can be observed where a lower dose of nanoparticles (25 μg/ml vs. 200 μg/l) resulted in less positive difference between treated and untreated wells suggesting that application of extracellular matrix protein may enhance endothelial cell growth in vitro. Cells were used immediately out of the freezer, a practice that can be unsuccessful due to a need for cells to accommodate to culture conditions. We also quantified the in vitro toxicity of free paclitaxol against bCA-EC's and human coronary smooth muscle cells (hCA-SMC's) plated on either laminin or tenascin (100 ng/well). Laminin has been shown to induce a more quiescent phenotype in vitro while tenascin is the major component of post-injury matrix in the medial artery (Flaherty et. al (1995); Lafleur et. al (1997); U.S. Pat. No. 6,124,60). Results are illustrated in FIG. 16B and indicate that endothelial cells plated on fibronectin are the most sensitive to paclitaxol of the four groups with an IC₅₀ for growth inhibition of about 15 μg/ml (solid line) compared to about 60 μg/ml for smooth muscle cells plated on tenascin (dashed line), a 4-fold increase. EC and SMC's on laminin were intermediate at about 45 μg/ml. Literature sources indicated a similar pattern in growth inhibition for coronary human endothelial cells vs. smooth muscle cells treated with rapamycin, an immunosuppressive agent between tested for inclusion in drug-eluting stents (Mohaci et. al (1997)). In quiescent wells, pretreated with increasing amounts of drug and challenged with growth factor addition, rapamycin's IC₅₀ for growth inhibition on coronary artery endothelial cells ranged from 0.1-1 nM, and from 1-10 nM for coronary artery smooth cells, a 10-fold increase. Direct and specific delivery of therapeutic agents to smooth muscle cells or optionally endothelial cells in the blood vessel wall offers increases usefulness of therapeutic strategies by limiting drug delivery and thus collateral damage to only target cells.

Applying s50 nanoparticles to a device for local delivery of nanoparticles may include the following steps:

(a) Compounding s50 nanoparticles containing therapeutic agent, preferably an antisense construct, into a binder comprised of a polymer and a disintegrant. The polymer is preferably a water-soluble polymer, protein, functional peptide equivalent or carbohydrate and many such polymers are known in the pharmaceutical art. A disintegrant is an agent that aids in rapid disintegration by increasing the solids percentage of the coating and thus decreasing its strength, such as a sugar, such as sucrose or trehalose. For a list of commonly acceptable polymers, binding agents and disintegrants, refer to the current Remingtons, the Handbook of Pharmaceutical Excipients, the United States Pharmacopeia and the Guide to Approved Excipients, located at http://www.fda.gov. Some acceptable polymeric coatings and matrices based on hydrogels are described in U.S. Pat. No. 5,593,974. More traditional polymeric coatings (biodegradable and non-biodegradable) and protein matrices for device drug delivery are described in U.S. Pat. Nos. 6,159,142, 6,258,121, 6,303,137, 6,143,037 and 6,342,250.

(b) Applying the compounded nanoparticles to the stent by dipping or spraying. Polymer compositions and methods for coating implants, especially sutures, are well-known in the art. Such coatings have been applied to surgical sutures to improve fiber lubricity, knot snug-down and tie-down performance, and for local delivery of pharmaceutical agents such as antibacterial agents. Methods for applying coatings to stents and other devices are well-know in the art and are described in U.S. Pat. Nos. 5,837,313, 6,159,142, 6,358,556 and 6,342,250. Stents may be prepared for coating as described in U.S. Pat. No. 6,120,847 and additional agents for prevention of thrombosis may be co-compounded or co-administered as described in U.S. Pat. No. 6,120,536.

(c) Optionally applying a seal coat to retard dissolution of the nanoparticle binding coat. In this way, a large bolus dose of nanoparticles can be released into the tissue. Seal coats are manufactured by cross-linking or additionally drying the binder comprised of the seal coat binder strength the coating and retard erosion by water-soluble fluids. Seal coating is well-known step is the design of oral dosage forms and is described in standard pharmacy texts such as the current Remington's and the Handbook of Pharmaceutical Dosage Forms.

(d) Optionally repeating this series of steps taking care that each coating is thoroughly dry or cured before proceeding to the next manufacturing step. In order to maintain adherence of the coating to the stent during, coating thickness should not exceed at or about 100 μm.

Other examples of disease states which may be treated be the described method include are pulmonary disorders such a acute respiratory distress syndrome, idiopathic pulmonary fibrosis, emphysema, primary pulmonary hypertension, cancer or proliferative or fibrotic nephropathies. In some of these situations, an appropriate optional route of local delivery could be an aerosol generated, e.g., from an inhaler or nebulizer.

### REFERENCES

All of the references, patents and applications cited herein above and below are incorporated by reference in there entirety as well as for the specifically described feature, if relevant.
Abra et. al (1981), Biochem. Biophys. Acta 666:493-503, “Liposome disposition in vivo: dose and vesicle-size effects.”
Akerman et. al (2002), PNAS 99(20):12617-12621, “Nanocrystal imaging in vivo.”
Bennet, M. (2003), Heart 89:218-224, “In-stent stenosis: pathology and implications for the development of drug-eluting stents.”
Brigger et. al, (2002), Adv. Drug Deliv. Rev. 54: 631-651, “Nanoparticles in cancer therapy and diagnosis.”
Bruchez, M. Jr., Moronne, M., Gin, P., Weiss, S., Alivisatos, A. P.: Semiconductor nanocrystals as fluorescent biological labels. Science 281(5385):2013-6, 1998.
Bulte, J., Douglas, T., Witwer, B., Zhang, S., Strable, E., Lewis, B., Zywicke, H., Miller, B., van Gelderen, P., Moskowitz, B., Duncan, I., Frank, J.: Magnetodendrimers allow endosomal magnetic labeling and in vivo tracking of stem cells. Nat Biotechnol 19(12):1141-7, 2001.
Chonn et. al, (1998) Adv. Drug Deliv. Reviews 30:73-83, “Recent advances in liposome technologies and their applications for systemic gene delivery.”
Chapman et. al, (1996), Ultramicroscopy 62:191-213, “A characterization of dark-field imaging of colloidal gold labels in a scanning transmission X-ray microscopy.”
Clarke et. al, (1998), Journal of Leukocyte Biology 63:163-168, “Myeloid-specific gene expression.”
Condon et al. (1996), Nature Med. 2(10): 1122-1127, “DNA-based immunization by in vivo transfection of dendritic cells.”
Dubertret et. al (2002), Science 298:1759-62, “In vivo imaging of quantum dots encapsulated in phospholipid micelles.”
Faust et. al (2000), Head Neck 22:341-346,″ Antisense oligonucleotides against PKCII-a inhibit growth of squamous cell carcinoma of the head and neck in vitro.
Flaherty et. al (1995), Mol Cell Biol 65:27a; Tech Bulletin #425, http://www.bdbio.com, “Phenotypic modulation of aortic smooth muscle cells using optimized cell culture environments.”
Grauer et. al (2002), Histochem Cell Biol 117:351-362, “Analysis of maturation states of rat bone marrow-derived dendritic cells using an improved culture techique.”
Frechet, J.: Functional polymers and dendrimers: reactivity, molecular architecture, and interfacial energy. Science 263(5154):1710-5, 1994.
Grayson, S., Frechet, J.: Convergent dendrons and dendrimers: from synthesis to applications. Chem Rev 101(12): 3819-68, 2001.
Guermonprez et. al (2002), Ann. Rev. Immunol. 20:621-27, “Antigen presentation and T cell stimulation by dendritic cells.”
Heath, J.: Fullerenes: C60's smallest cousin. Nature 393: 730-1, 1998.
Hunt et. al, (1992), J. Biomed. Mat. Res. 26: 819-828, “Stimulation of neutrophil movement by metal ions.”
Inaba et. al (1995), Cell Immunol. 163:148-156, “Tissue distribution of the DEC-205 protein that is detected by the monoclonal antibody NLDC-145.”
Johansen et. al, (2000)). Eur J. Pharm. Biopharm. 50:129-146, “Revisiting PLA/PLGA microspheres: an analysis of their potential in parenteral vaccination.”
Kreuter et. al (1996), J. Anat. 189:503-505, “Nanoparticles and microparticles for drug and vaccine delivery.”
Krieg et. al (2002), Ann Rev. Immunol. 20:700-60, “CpG motifs in bacterial DNA and their immune effects”.
Lafleur et. al, (1997), J. Biol. Chem. 272(52):32798-32803, “Aortic smooth muscle cells interact with tenascin-C through its fibrinogin-like domain.”
Larregina et. al (1997), Immunol. 91; 303-13, “Pattern of cytokine receptors expressed by human dendritic cells migrated from dermal explants.”
Lewin, M., Carlesso, N., Tung, C., Tang, X., Cory, D., Scadden, D., Weissleder, R.: Tat peptide-derivated magnetic nanoparticles allow in vivo tracking and recovery of progenitor cells. Nat Biotechnol, 18(4):410-4, 2000.
Lipscomb et. al (2001), Physiol. Rev. 82:97-130, “Dendritic cells: immune regulators in health and disease.”
Manome et. al (1998), Immunol. 98:481-490, “Simple chemicals can induce maturation and apoptosis of dendritic cells.”
Micic, O., Cheong, H., Fu, H., Zunger, A., Sprague, J., Mascarenhas, A., and Nozik, A.: Size-dependent spectroscopy of InP quantum dots. J. Phys. Chem. B., 101(25):4904-12, 1997.
Moingeon (2002), J. Biotech. 98:189-98, “Strategies for designing vaccines eliciting Th1 responses in humans.”
Mohacsi et. al (1997), J Heart Lung Transplant 16:484-92, “Different inhibitory effects of immunosuppressive drugs on human and rat aortic smooth muscle and endothelial proliferation stimulated by PDGF or ECGF.”
Nie, S., and Emory, S. R.: Probing single molecules and single nanoparticles by surface-enhanced raman scattering. Science 275(5303):1102-6, 1997.
Normal et. al (2000), Gene Ther. 7:1425-1430, “Liposome-mediated, nonviral gene transfer induces a systemic inflammatory response.”
Reiss et. al (1997), Breast Cancer Research & Treatment 45(1):81-95, “Transforming growth factor-beta in breast cancer, a working hypothesis.”
Rudginsky et. al (2001), Gene Ther. 4(4):347-355), “Antitumor activity of cationic lipid complexed with immunostimulatory DNA”
Schreder, T., Schmidt, V., Ptatschek, U., Winkler, A., Materny, E., Umbach, M., Lerch, G., Muller, W., Kiefer, W., and Spanhel, L.: CdTe/CdS clusters with “core shell” structure in colloids and films: The path of formation and thermal breakup. J. Phys. Chem. B., 104(8):1677-85, 2000.
Sasaki et. al (2001), Nature Biotech 19: 543-47, “Apoptosis-mediated enhancement of DNA-raised immune responses by mutant caspases.”
Singh et. al (2002), Pharm Res. 19(6):715-28, “Recent advances in vaccine adjuvants.”
Storm et. al (1998), Clin. Can. Res. 3: 111-15, “Doxorubicin entrapped in sterically stabilized liposomes: effects on bacterial blood clearance capacity of the mononuclear phagocyte system.”
Tousignant et. al (2000), Hu. Gene Ther. 11: 2493-2513, “Comprehensive analysis of the acute toxicities induced by systemic administration of cationic lipid:plasmid DNA complexes in mice.”
Unger, G., Adams, G., Davis, A., Ahmed, K., (2002) AACR Proceedings, 43: 577, “Effective chemotherapeutic activity by sub50-nm nanocapsule antisense to protein kinase CK2 for eradication of in vitro tumor nests via targeted caveolar-mediated endocytosis.”
Unger, G., Slayton, J., Adams, G., Davis, A., Ahmed, K., (2003) AACR Proceedings, 44: 1473, “Antisense formulation via sub50-nm nanoencapsulation enhances effectiveness of problematic medicinal chemistry for tumor-specific antisense to PK CK2.”
Virmani, R. et. al, (2002), Circulation 106: 2649-2651, “Mechanism of late in-stent restenosis after implantation of a paclitaxel derivative-eluting polymer stent system in humans.”
Weeratna et. al (2000), Intervirology 43:2128-226, “Optimization strategies for DNA vaccines”.

1. A collection of particles comprising
an agent, a surfactant molecule having an HLB value of less than about 6.0 units, and a polymer, wherein the collection of particles has an average diameter of less than about 100 nanometers as measured by atomic force microscopy of a plurality of the particles following drying of the particles, wherein the agent comprised a member of the group consisting of proteins, carbohydrates, polypeptides, adjuvants, nucleic acids encoding a protein, visualization agents, and markers.

2. The collection of particles of claim 1 wherein the agent comprises a nucleic acid disposed in a vector.

3. The collection of particles of claim 2 wherein the vector encodes a member of the group consisting of green fluorescent protein, beta galactosidase, and a bacterial protein.

4. The collection of particles of claim 1 wherein the agent and the polymer soluble in aqueous solution are the same material.

5. The collection of particles of claim 1 wherein the adjuvant comprises of Freund's adjuvant, Corynebacterium parvum, bacterial antigens, histamine, interferon, transfer factor, tuftsin, interleukin-1 nickel, Montanide ISA, Ribi Adjuvant System, Syntex Adjuvant Formulation, aluminum salts, or GerbuR adjuvant.

6. The collection of particles of claim 1 wherein the protein comprises an immune system danger signal or a dendritic cell maturation factor.

7. The collection of particles of claim 1 wherein the protein comprises an antibody.

8. The collection of particles of claim 1 wherein the particles comprise a ligand for targeting a selected cell type.

9. The collection of particles of claim 8 wherein the ligand is bindable to dendritic cells.

10. The collection of particles of claim 9 wherein the ligand bindable to dendritic cells is E-selectin.

11. A kit comprising the collection of particles of claim 1 and instructions for using the collection of particles.

12. A kit comprising the collection of particles of claim 2 and instructions for using the collection of particles.

13. A method of delivering an agent to an antigen presenting cell, the method comprising exposing a cell to a collection of particles that comprises an agent, a surfactant molecule having an HLB value of less than about 6.0 units, a polymer, and a ligand that binds to an antigen presenting cell, wherein the collection of particles has an average diameter of less than about 100 nanometers as measured by atomic force microscopy of a plurality of the particles following drying of the particles.

14. The method of claim 13 wherein the ligand is E-selectin.

15. The method of claim 13 wherein the agent comprises a member of the group consisting of proteins, carbohydrates, polypeptides, adjuvants, nucleic acids encoding an antigen, visualization agents, and markers.

16. The method of claim 13 wherein the agent comprises a vector that encodes for green fluorescent protein or betagalactosidase.

17. The method of claim 13 wherein the agent comprises a vector that encodes for a bacterial protein.

18. The method of claim 13 wherein the adjuvant comprises of Freund's adjuvant, Corynebacterium parvum, bacterial antigens, histamine, interferon, transfer factor, tuftsin, interleukin-1 nickel, Montanide ISA, Ribi Adjuvant System, Syntex Adjuvant Formulation, aluminum salts, or GerbuR adjuvant.

19. A method of affecting function of a cell, the method comprising exposing the cell to an agent that inhibits protein kinase 2 function.

20. The method of claim 19 wherein the agent comprises an antisense molecule that inhibits the expression of a member of the group consisting of protein kinase 2 alpha, protein kinase 2 alpha prime, and protein kinase 2 beta.

21. The method of claim 20 wherein the antisense molecule avoids binding to a start codon.

22. The method of claim 19 wherein the agent is an antisense molecule.

23. The method of claim 19 wherein the agent is combined with a surfactant and a polymer in a nanoparticle of less than about 100 nm in diameter.

24. A collection of particles comprising: an agent, a surfactant molecule having an HLB value of less than about 6.0 units, and a polymer, wherein the collection of particles has an average diameter of less than about 200 nanometers as measured by atomic force microscopy of a plurality of the particles following drying of the particles, wherein the agent is an imaging agent.

25. The collection of particles of claim 24 wherein the imaging agent is a member of the group consisting of stains, vital dyes, fluorescent markers, radioactive markers, enzymes and plasmid constructs encoding markers, enzymes and combinations thereof.

26. The collection of particles of claim 24 wherein the imaging agent is visualized after it is taken up intracellularly by a cell.

27. The collection of particles of claim 24 wherein the imaging agent is an agent that provides a signal when integrated by a technique selected from the group consisting of magnetic resonance imaging, radionuclide imaging, computed tomography, ultrasound, and optical imaging.

28. The collection of particles of claim 24 wherein the imaging agent is a member of the group consisting of fluorescent molecules, antibodies, avidin, biotin, colloidal metals, gold, silver, reporter enzymes, horseradish peroxidase, superparamagnetic transferrin, second reporter systems, tyrosinase, and paramagnetic chelates.

29. The collection of particles of claim 24 wherein the imaging agent is a peptide specific to a molecule, cell type, or tissue type.

30. The collection of particles of claim 24 wherein the imaging agent comprises an antibody.

31. The collection of particles of claim 24 further comprising a targeting molecule that is bindable to a target molecule.

32. The collection of particles of claim 31 wherein the targeting molecule is a ligand for a cell surface receptor.

33. The collection of particles of claim 31 wherein the target molecule is a cell surface receptor.

34. A method of delivering an agent to a cell, the method comprising:
exposing a cell to a collection of particles that comprises an imaging agent, a surfactant molecule having an HLB value of less than about 6.0 units, and a polymer, wherein the collection of particles has an average diameter of less than about 100 nanometers as measured by atomic force microscopy of a plurality of the particles following drying of the particles.

35. The method of claim 34 wherein the imaging agent is a member of the group consisting of stains, vital dyes, fluorescent markers, radioactive markers, enzymes and plasmid constructs encoding markers or enzymes, fluorescent molecules, antibodies, avidin, biotin, colloidal metals, gold, silver, reporter enzymes, horseradish peroxidase, superparamagnetic transferrin, second reporter systems, tyrosinase, paramagnetic chelates and combinations thereof.

36. The method of claim 34 wherein the imaging agent is visualized after it is taken up intracellularly by a cell.

37. The method of claim 34 further comprising forming an image form the imaging agent with a technique selected from the group consisting of magnetic resonance imaging, radionuclide imaging, computed tomography, ultrasound, and optical imaging.

38. The collection of particles of claim 34 wherein the imaging agent comprises an antibody or a peptide specific to a molecule, cell type, or tissue type.

39. A kit comprising a collection of particles comprising: an agent, a surfactant molecule having an HLB value of less than about 6.0 units, and a polymer, wherein the collection of particles has an average diameter of less than about 200 nanometers as measured by atomic force microscopy of a plurality of the particles following drying of the particles, wherein the agent is an imaging agent, with the kit further comprising instructions for using the collection of particles.

40. The kit of claim 39 wherein the imaging agent is a member of the group consisting of stains, vital dyes, fluorescent markers, radioactive markers, enzymes and plasmid constructs encoding markers or enzymes, fluorescent molecules, antibodies, avidin, biotin, colloidal metals, gold, silver, reporter enzymes, horseradish peroxidase, superparamagnetic transferrin, second reporter systems, tyrosinase, paramagnetic chelates and combinations thereof.

41. The kit of claim 39 wherein the imaging agent is visualized after it is taken up intracellularly by a cell.

42. A method of delivering an agent to a cancer cell, the method comprising: exposing a cancer cell to a collection of particles that comprises an agent, a surfactant molecule having an HLB value of less than about 6.0 units, a polymer, an adjuvant, and a ligand that targets to the cancer cell, wherein the collection of particles has an average diameter of less than about 100 nanometers as measured by atomic force microscopy of a plurality of the particles following drying of the particles.

43. The method of claim 42 wherein the adjuvant is chosen from the group consisting of Freund's adjuvant, Corynebacterium parvum, bacterial antigens, histamine, interferon, transfer factor, tuftsin, interleukin-1 nickel, Montanide ISA, Ribi Adjuvant System, Syntex Adjuvant Formulation, aluminum salts, and GerbuR adjuvant and combination thereof.

44. The method of claim 42 wherein the ligand is tenascin.

45. The method of claim 42 wherein the agent is a member of the group consisting of stains, vital dyes, fluorescent markers, radioactive markers, enzymes and plasmid constructs encoding markers or enzymes, fluorescent molecules, antibodies, avidin, biotin, colloidal metals, gold, silver, reporter enzymes, horseradish peroxidase, superparamagnetic transferrin, second reporter systems, tyrosinase, paramagnetic chelates and combinations thereof.

46. The method of claim 42 wherein the agent is a member of the group consisting of toxins, apoptotic agents, antisense molecules, bacterial proteins and combinations thereof.

47. The method of claim 46 wherein the agent is an antisense molecule that inhibits the expression of protein kinase 2.

48. A collection of coated particles comprising particles and a coating, the coating comprising a binder and the particles comprising an agent, a surfactant molecule having an HLB value of less than about 6.0 units, and a polymer, wherein the collection of particles has an average diameter of less than about 100 nanometers as measured by atomic force microscopy of a plurality of the particles following drying of the particles.

49. The coating of claim 48 wherein the agent is a member of the group consisting of antigenic proteins, adjuvants, nucleic acids encoding an antigen, visualization agents, and markers and combinations thereof.

50. The coating of claim 48 wherein the agent is a member of the group consisting of stains, vital dyes, fluorescent markers, radioactive markers, enzymes and plasmid constructs encoding markers or enzymes, fluorescent molecules, antibodies, avidin, biotin, colloidal metals, gold, silver, reporter enzymes, horseradish peroxidase, superparamagnetic transferrin, second reporter systems, tyrosinase, paramagnetic chelates, bacterial proteins and combinations thereof.

51. The coating of claim 48 wherein the agent is a member of the group consisting of toxins, apoptotic agents, and antisense molecules.

52. The coating of claim 48 wherein the agent is an antisense molecule that inhibits the expression of a member of the group consisting of protein kinase 2 alpha, protein kinase 2 alpha prime, and protein kinase 2 beta.

53. A biocompatible stent coated with the collection of particles of claim 50.

54. A biocompatible stent coated with the collection of particles of claim 51.

55. The stent of claim 54 wherein the polymer comprises vinylpyrrolidone.

56. A method of coating a collection of particles, the method comprising mixing a binder with a collection of particles comprising an agent, a surfactant molecule having an HLB value of less than about 6.0 units, and a polymer, wherein the collection of particles has an average diameter of less than about 100 nanometers as measured by atomic force microscopy of a plurality of the particles following drying of the particles.

57. The method of claim 56 further comprising applying the coating to a stent by dipping or spraying.

58. The method of claim 56 further comprising mixing a disintegrant with the collection of nanoparticles or binder.

59. The method of claim 58 further comprising applying a sealing layer on the mixture of the binder and the collection of particles to retard dissolution of the mixture of the binder and the collection of particles.


SEQUENCE LISTING (3 sequences)

SEQ ID NO: 1 - DNA, 1677 bp, Homo sapiens
tgtcacccaggctggagtgcagtggcgcaatctcagctcactgcaacctccacctccctggttcaagcgattctcctgcctcctccgcccgacgccccgcgtcccccgccgcgccgccgccgccaccctctgcgccccgcgccgccccccggtcccgcccgccatgcccggcccggccgcgggcagcagggcccgggtctacgccgaggtgaacagtctgaggagccgcgagtactgggactacgaggctcacgtcccgagctggggtaatcaagatgattaccaactggttcgaaaacttggtcggggaaaatatagtgaagtatttgaggccattaatatcaccaacaatgagagagtggttgtaaaaatcctgaagccagtgaagaaaaagaagataaaacgagaggttaagattctggagaaccttcgtggtggaacaaatatcattaagctgattgacactgtaaaggaccccgtgtcaaagacaccagctttggtatttgaatatatcaataatacagattttaagcaactctaccagatcctgacagactttgatatccggttttatatgtatgaactacttaaagctctggattactgccacagcaagggaatcatgcacagggatgtgaaacctcacaatgtcatgatagatcaccaacagaaaaagctgcgactgatagattggggtctggcagaattctatcatcctgctcaggagtacaatgttcgtgtagcctcaaggtacttcaagggaccagagctcctcgtggactatcagatgtatgattatagcttggacatgtggagtttgggctgtatgttagcaagcatgatctttcgaagggaaccattcttccatggacaggacaactatgaccagcttgttcgcattgccaaggttctgggtacagaagaactgtatgggtatctgaagaagtatcacatagacctagatccacacttcaacgatatcctgggacaacattcacggaaacgctgggaaaactttatccatagtgagaacagacaccttgtcagccctgaggccctagatcttctggacaaacttctgcgatacgaccatcaacagagactgactgccaaagaggccatggagcacccatacttctaccctgtggtgaaggagcagtcccagccttgtgcagacaatgctgtgctttccagtggtctcacggcagcacgatgaagactggaaagcgacgggtctgttgcggttctcccacttttccataagcagaacaagaaccaaatcaaacgtcttaacgcgtatagagagatcacgttccgtgagcagacacaaaacggtggcaggtttggcgagcacgaactagaccaagcgaagggcagcccaccaccgtatatcaaacctcacttccgaatgtaaaaggctcacttgcctttggcttcctgttgacttcttcccgacccagaaagcatggggaatgtgaagggtatgcagaatgttgttggttactgttgctccccgagcccctcaactcgtcccgtggccgcctgtttttccagcaaaccacgctaactagctgaccacagactccacagtggggggacgggcgcagtatgtggcatggcggcagttacatattattattttaaaagtatatattattgaataaaaggttttaaaag

SEQ ID NO: 2 - DNA, 1128 bp, Homo sapiens
gcttctcgttgtgccccgcccgcaagcgccctcctccgggccttcgtgacagccaggtcgtgcgcgggtcatcctgggattggtagttcgctttctctcatttagccagtttctttctctaccggggactccgtgtcccggcatccaccgcggcacctgacccttggcgcttgcgtgttgccctcttccccaccctccctaatttccactccccccaccccacttcgcctgccgcggtcgggtccgcggcctgcgctgtagcggtcgccgccgttccctggaagtagcaacttccctaccccaccccagtcctggtccccgtccagccgctgacgtgaagatgagcagctcagaggaggtgtcctggatttcctggttctgtgggctccgtggcaatgaattcttctgtgaagtggatgaagactacatccaggacaaatttaatcttactggactcaatgagcaggtccctcactaccgacaagctctagacatgatcttggacctggagcctgatgaagaactggaagacaaccccaaccagagtgacctgattgagcaggcagccgagatgctttatggattgatccacgcccgctacatccttaccaaccgtggcatcgcccagatgttggaaaagtaccagcaaggagactttggttactgtcctcgtgtgtactgtgagaaccagccaatgcttcccattggcctttcagacatcccaggtgaagccatggtgaagctctactgccccaagtgcatggatgtgtacacacccaagtcatcaagacaccatcacacggatggcgcctacttcggcactggtttccctcacatgctcttcatggtgcatcccgagtaccggcccaagagacctgccaaccagtttgtgcccaggctctacggtttcaagatccatccgatggcctaccagctgcagctccaagccgccagcaacttcaagagcccagtcaagacgattcgctgattccctcccccacctgtcctgcagtctttgacttttcctttcttttttgccaccctttcaggaaccctgtatggtttttagtttaaattaaaggagtcgttattgtggtgggaatatgaaataaagtagaagaaaaggcc

SEQ ID NO: 3 - DNA, 2323 bp, Homo sapiens
cccgcctcctggtaggagggggtttccgcttccggcagcagcggctgcagcctcgctctggtccctgcggctggcggccgagccgtgtgtctcctcctccatcgccgccatattgtctgtgtgagcagaggggagagcggccgccgccgctgccgcttccaccacagtttgaagaaaacaggtctgaaacaaggtcttacccccagctgcttctgaacacagtgactgccagatctccaaacatcaagtccagctttgtccgccaacctgtctgacatgtcgggacccgtgccaagcagggccagagtttacacagatgttaatacacacagacctcgagaatactgggattacgagtcacatgtggtggaatggggaaatcaagatgactaccagctggttcgaaaattaggccgaggtaaatacagtgaagtatttgaagccatcaacatcacaaataatgaaaaagttgttgttaaaattctcaagccagtaaaaaagaagaaaattaagcgtgaaataaagattttggagaatttgagaggaggtcccaacatcatcacactggcagacattgtaaaagaccctgtgtcacgaacccccgccttggtttttgaacacgtaaacaacacagacttcaagcaattgtaccagacgttaacagactatgatattcgattttacatgtatgagattctgaaggccctggattattgtcacagcatgggaattatgcacagagatgtcaagccccataatgtcatgattgatcatgagcacagaaagctacgactaatagactggggtttggctgagttttatcatcctggccaagaatataatgtccgagttgcttcccgatacttcaaaggtcctgagctacttgtagactatcagatgtacgattatagtttggatatgtggagtttgggttgtatgctggcaagtatgatctttcggaaggagccatttttccatggacatgacaattatgatcagttggtgaggatagccaaggttctggggacagaagatttatatgactatattgacaaatacaacattgaattagatccacgtttcaatgatatcttgggcagacactctcgaaagcgatgggaacgctttgtccacagtgaaaatcagcaccttgtcagccctgaggccttggatttcctggacaaactgctgcgatatgaccaccagtcacggcttactgcaagagaggcaatggagcacccctatttctacactgttgtgaaggaccaggctcgaatgggttcatctagcatgccagggggcagtacgcccgtcagcagcgccaatatgatgtcagggatttcttcagtgccaaccccttcaccccttggacctctggcaggctcaccagtgattgctgctgccaacccccttgggatgcctgttccagctgccgctggcgctcagcagtaacggccctatctgtctcctgatgcctgagcagaggtgggggagtccaccctctccttgatgcagcttgcgcctggcggggaggggtgaaacacttcagaagcaccgtgtctgaaccgttgcttgtggatttatagtagttcagtcataaaaaaaaaattataataggctgattttcttttttctttttttttttaactcgaacttttcataactcaggggattccctgaaaaattacctgcaggtggaatatttcatggacaaatttttttttctcccctcccaaatttagttcctcatcacaaaagaacaaagataaaccagcctcaatcccggctgctgcatttaggtggagacttcttcccattcccaccattgttcctccaccgtcccacactttagggggttggtatctcgtgctcttctccagagattacaaaaatgtagcttctcaggggaggcaggaagaaaggaaggaaggaaagaaggaagggaggacccaatctataggagcagtggactgcttgctggtcgcttacatcactttactccataagcgcttcagtggggttatcctagtggctcttgtggaagtgtgtcttagttacatcaagatgttgaaaatctacccaaaatgcagacagatactaaaaacttctgttcagtaagaatcatgtcttactgatctaaccctaaatccaactcatttatacttttatttttagttcagtttaaaatgttgataccttccctcccaggctccttaccttggtcttttccctgttcatctcccaacatgctgtgctccatagctggtaggagagggaaggcaaaatctttcttagttttctttgtcttggccattttgaattc